SEQUENCE LISTING (2 sequences)

SEQ ID NO: 1 - DNA, 25 bp, artificial
  primer
ggggattggttttgacgtttttgcg

SEQ ID NO: 2 - DNA, 30 bp, artificial
  primer
aagcattaaataaagcgaatacatccttat

(19) United States
(12) Patent Application Publication
(10) Pub. No.: US 2005/0222048 A1
(43) Pub. Date: Oct. 6, 2005

(54) Novel synthetic C-glycolipids, their synthesis and use to treat infections, cancer and autoimmune diseases

(75) Inventors: Moriya Tsuji, New York, NY (US); Richard Franck, Riverside, CT (US); Guangwu Chen, Corona, NY (US)

Correspondence Address: DARBY & DARBY P.C., P. O. BOX 5257, NEW YORK, NY, 10150-5257, US

(73) Assignees: The Research Foundation of The City University of New York, New York, NY (US); New York University, New York, NY (US)

(21) Appl. No.: 11/096,340

(22) Filed: Mar. 31, 2005

Related U.S. Application Data
(60) Provisional application No. 60/558,467, filed on Mar. 31, 2004.

(51) Int. Cl. A61K 31/7008

(52) U.S. Cl. 514/23; 536/53

(57) ABSTRACT

The invention is directed to novel compounds of formulae (I), (II) and (III): 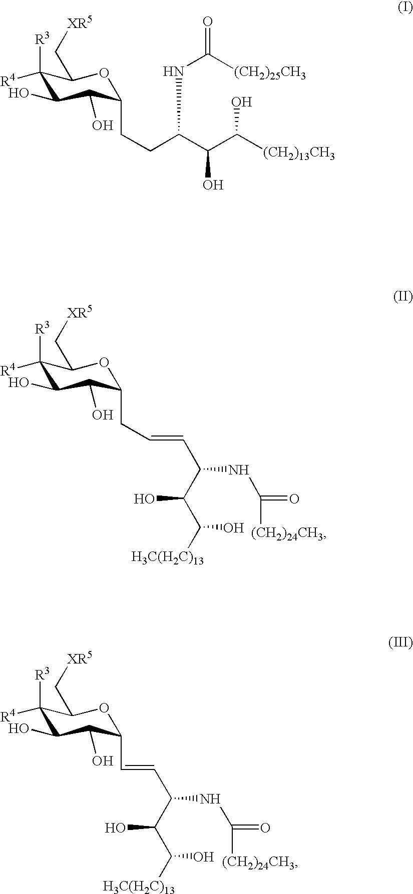 wherein X is O or NH;
R³ is OH or a monosaccharide and R⁴ is hydrogen, or R³ is hydrogen and R⁴ is OH or a monosaccharide;
R⁵ is hydrogen or a monosaccharide; and pharmaceutically acceptable salts or esters thereof. The invention is also directed to the use of the compounds both directly and as immune adjuvants for treating cancer, infectious diseases and autoimmune diseases. The invention is also directed to syntheses of the intermediates which can be used to make these novel compounds.

This invention was made with government support under grant number R21 AI47840-01A1, awarded by the National Institute of Health/National Institute of Allergy and Infectious Diseases, and grant number R01 GM 60271, awarded by the National Institute of Health/General Medical Sciences. Accordingly, the United States Government has certain rights in the invention.

### FIELD OF THE INVENTION

The invention is directed to novel synthetic C-glycolipids which are useful to treat infections, cancer and autoimmune diseases (both directly and as adjuvants via augmenting the immunogenicity of various antigens). Methods of making such novel synthetic C-glycolipids are also disclosed.

### BACKGROUND OF THE INVENTION

Glycolipids are molecules typically found in plasma membranes of animal and plant cells. Glycolipids contain an oligosaccharide which is bonded to a lipid component. Sphingoglycolipids are complex glycolipids which contain ceramide as the lipid component. One class of sphingoglycolipids is alpha-galactosylceramides (α-GalCer), which contain D-galactose as the saccharide moiety, and ceramide as the lipid moiety.

Various α-GalCer compounds have been shown in the prior art. U.S. Pat. No. 5,780,441 describes mono- and di-glycosylated α-GalCer compounds of the following structure: 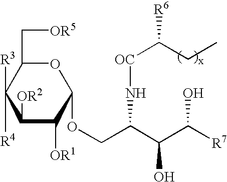
wherein R¹ is H or 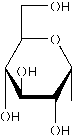
R² is H, 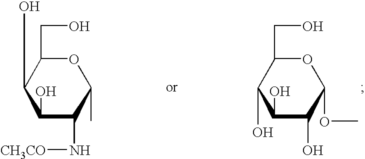
R³ and R⁶ are H or OH, respectively,
R is H, OH or 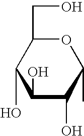
R⁵ is H or 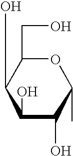
x is an integer from 19 to 23; and
R⁷ is —(CH₂)₁₁—CH₃, —(CH₂)₁₂—CH₃, —(CH₂)₁₃—CH₃, —(CH₂)₉—CH(CH₃)₂, —(CH₂)₁₀—CH(CH₃)₂, —(CH₂)₁₁—CH(CH₃)₂, —(CH₂)₁₁—-CH(CH₃)—C₂H₅,
wherein at least one of R¹, R², R⁴ and R⁵ is a glycosyl moiety.

α-GalCer can be extracted from Okinawan marine sponges (Natori et al., Tetrahedron, 50: 2771-2784, 1994) or its synthetic analog, KRN 7000 [(2S,3S,4R)-1-O-(α-D-galactopyranosyl)-2-(N-hexacosanoylamino)-1,3,4,-octadecanetriol], can be obtained from Pharmaceutical Research Laboratories, Kirin Brewery (Gumna, Japan) or synthesized as described previously (see, e.g., Morita et al., J. Med. Chem., 1995, 38: 2176-2187; Kobayashi et al., 1995, Oncol. Res., 7:529-534; Kawano et al., 1997, Science, 278:1626-9; Burdin et al., 1998, J. Immunol., 161:3271; Kitamura et al., 1999, J. Exp. Med., 189:1121; U.S. Pat. No. 5,936,076).

KRN 7000 has the structure:

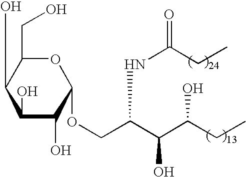

U.S. Pat. No. 6,635,622 discloses compounds of the formula: 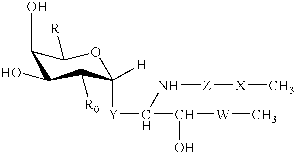
wherein W represents carbon chain from 9 to 17 which containing double bond or hydroxy group occasionally; X represents carbon chain from 11 to 25 which containing double bond or hydroxy group occasionally; Y represents (CH₂)ₐ—CH═CH—(CH₂)_{a′}—, —(CH₂)ₐ′ (a, a′ denotes an integer of 0-5 and a+a′ is 5 and under), —S(O)₀₋₂ CH₂—, —NHCH₂—; Z represents —CO—, —SO₂—; R represents —CH₂OH, —CO₂H, —CH₂OCH₂CO₂H, —CH₂OSO₃H; R₀ represents —OH, —NH₂, —NHAc. It discloses that such compounds can be made using Wittig reactions.

Co-pending commonly owned U.S. patent application Ser. No. 10/462,211 discloses novel C-glycolipid compounds of the formula: 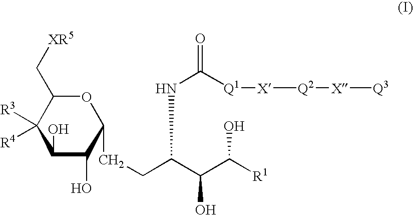
wherein X is O or NH;
R¹ is selected from the group consisting of —(CH₂)₁₁CH₃, —(CH₂)₁₂CH₃, —(CH₂)₁₃CH₃, —(CH₂)₉CH(CH₃)₂, —(CH₂)₁₀CH(CH₃)₂, —(CH₂)₁₁CH(CH₃)₂ and ₉(CH₂)₁₁CH(CH₃)—C₂H₅;
R³ is OH or a monosaccharide and R⁴ is hydrogen, or R³ is hydrogen and R⁴ is OH or a monosaccharide;
R⁵ is hydrogen or a monosaccharide;
Q¹ is optionally present and is a C₁₋₁₀ straight or branched chain alkylene, alkenylene, or alkynylene;
X′ is optionally present and is O, S or NR⁸;
Q² is optionally present and is a C₁₋₁₀ straight or branched chain alkylene, alkenylene or alkynylene;
X″ is optionally present and is O, S or NR⁸;
Q³ is a straight or branched chain C₁₋₁₀ alkyl, alkenyl or alkynyl, or is hydrogen,
wherein each Q¹, Q² or Q³ is optionally substituted with hydroxyl, halogen, cyano, nitro, SO₂, NHR⁸, or C(═O)—R⁹; and wherein
   R⁸ is hydrogen, C₁₋₅ alkyl, C₁₋₅ alkoxy, halogen, cyano, nitro, SO₂ or C(═O)—R⁹;
   R⁹ is hydrogen, C₁₋₅ alkyl, C₁₋₅ alkoxy or NHR¹⁰;
   R¹⁰ is hydrogen, C₁₋₅ alkyl or C₁₋₅ alkoxy.

The co-pending '211 application also discloses a compound of the formula: 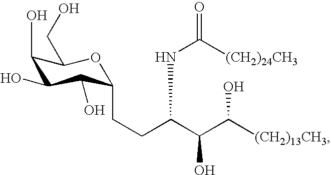
which is also known as CRONY 101.

α-GalCer and KRN 7000 have been described as immunostimulating agents effective to treat cancer, infections and autoimmune diseases (Kakimi, J. Exp. Med. 192: 921-930 (2000); Gonzalez-Asequinaloza, Proc. Natl. Acad. Sci. USA 97: 8461-8466 (2000); Sharif, Nature Medicine 7: 1057-1062 (2001); Hong, Nature Medicine 9: 1052-1056 (2001); Kawakami, Infection and Immunity 69: 213-220 (2001); Miyamoto, Nature 413: 531-534 (2001); Kobayashi, et al., Oncol. Res. 7:529-534 (1995); Nakagawa, Canc. Res. 58, 1202-1207 (1998); Kawano et al., 1997, Science, 278:1626-9, Burdin et al., 1998, J. Immunol., 161:3271; Kitamura et al., J. Exp. Med., 1999, 189: 1121).

The successful elimination of pathogens, neoplastic cells, or self-reactive immune mechanisms following prophylactic or therapeutic immunization depends to a large extent on the ability of the host's immune system to become activated in response to the immunization and mount an effective response, preferably with minimal injury to healthy tissue.

The immunogenicity of a relatively weak antigen can be enhanced by the simultaneous or more generally conjoined administration of the antigen with an “adjuvant”, usually a substance that is not immunogenic when administered alone, but will evoke, increase and/or prolong an immune response to an antigen. In the absence of adjuvant, reduced or no immune response may occur, or worse the host may become tolerized to the antigen. In the design of effective vaccines, immunological adjuvants serve as critical components, which accelerate, prolong, and/or enhance an antigen-specific immune response as well as provide the selective induction of the appropriate type of response.

Adjuvants can be found in a group of structurally heterogeneous compounds (Gupta et al., 1993, Vaccine, 11:293-306). Classically recognized examples of adjuvants include oil emulsions (e.g., Freund's adjuvant), saponins, aluminum or calcium salts (e.g., alum), non-ionic block polymer surfactants, lipopolysaccharides (LPS), mycobacteria, tetanus toxoid, and many others. Theoretically, each molecule or substance that is able to favor or amplify a particular situation in the cascade of immunological events, ultimately leading to a more pronounced immunological response can be defined as an adjuvant.

Although little is known about their mode of action, it is currently believed that adjuvants augment immune responses by one of the following mechanisms: (1) increasing the biological or immunologic half-life of antigens (see, e.g., Lascelles, 1989, Vet. Immunol. Immunopathol., 22: 15-27; Freund, 1956, Adv. Tuber. Res., 7: 130-147); (2) improving antigen delivery to antigen-presenting cells (APCs), as well as antigen processing and presentation by the APCs (see, e.g., Fazekas de St. Groth et al., Immunol. Today, 19: 448-454, 1998), e.g., by enabling antigen to cross endosomal membranes into the cytosol after ingestion of antigen-adjuvant complexes by APCs (Kovacsovics-Bankowski et al., Science, 1995, 267: 243-246); (3) mimicking microbial structures leading to improved recognition of microbially-derived antigens by the pathogen-recognition receptors (PRRs), which are localized on accessory cells from the innate immune system (Janeway, 1989, Cold Spring Harbor Symp. Quant. Biol., 54:1-13; Medzhitov, 1997, Cell, 91:295-298; Rook, 1993, Immunol. Today, 14:95-96); (4) mimicking danger-inducing signals from stressed or damaged cells which serve to initiate an immune response (see, e.g., Matzinger, 1994, Annu. Rev. Immunol., 12:991-209), (5) inducing the production of immunomodulatory cytokines (see, e.g., Nohria, 1994, Biotherapy, 7:261-269; Iwasaki et al., 1997, J. Immunol., 158:4591-4601; Maecker et al., 1997, Vaccine, 15:1687-1696); (6) biasing the immune response towards a specific subset of the immune system (e.g., generating Th1- or Th2-polarized response, etc.) (Janssen et al., Blood, 97:2758-2763, 2001; Yamamoto et al., Scand. J. Immunol., 53:211-217, 2001; Weiner G. J., J. Leukoc. Biol., 68:455-63, 2000; Lucey, Infect. Dis. Clin. North Am., 13:1-9, 1999), and (7) blocking rapid dispersal of the antigen challenge (the “depot effect”) (Hood et al., Immunology, Second Ed., 1984, Benjamin/Cummings: Menlo Park, Calif.; St Clair et al., Proc. Natl. Acad. Sci. U.S.A., 96:9469-9474, 1999; Ahao et al., J. Pharm. Sci., 85:1261-1270, 1996; Morein et al., Vet. Immunol. Immunopathol., 54:373-384, 1996). (See also reviews by Schijns, Curr. Opin. Immunol., 12: 456-463, 2000; Vogel, Clin. Infect. Dis., 30 [Suppl. 3]: S266-70, 2000; Singh and O'Hagan, Nature Biotechnol., 17: 1075-81, 1999; Cox and Coulter, Vaccine, 15: 248-256, 1997).

Recent observations strongly suggest that endogenously produced cytokines act as essential communication signals elicited by traditional adjuvants (Brewer et al., 1996, Eur. J. Immunol., 26:2062-2066; Smith et al., 1998, Immunology, 93:556-562; Allison, Dev. Biol. Stand., 1998, 92:3-11; Unkeless, Annu. Rev. Immunol., 1988, 6:251-81; Phillips et al., Vaccine, 1992, 10:151-8).

The benefit of incorporating adjuvants into vaccine formulations to enhance immunogenicity must be weighed against the risk that these agents will induce adverse local and/or systemic reactions. Thus, many potent immunoadjuvants, such as Freund's Complete or Freund's Incomplete Adjuvant, are toxic and are therefore useful only for animal research purposes, not human vaccinations. Currently, aluminum salts and MF59 are the only vaccine adjuvants approved for human use. The development of more potent and less toxic novel adjuvants may allow novel vaccines to be developed and both novel and existing vaccines to be used as therapeutic as well as improved prophylactic agents.

Recently, a novel lymphoid lineage, natural killer T (NKT) cells, distinct from mainstream T cells, B cells and NK cells, has been identified (Arase et al., 1992, Proc. Natl. Acad. Sci. USA, 89:6506; Bendelac et al., 1997, Annu. Rev. Immunol., 15:535). These cells are therefore implicated as key effector cells in innate immune responses and potentially as important participants in the development of adaptive immune responses.

Recently, it was demonstrated that NKT cells can be activated both in vitro and in vivo by α-GalCer extracted from Okinawan marine sponges or its synthetic analog KRN 7000. Thus, it was shown that α-GalCer can stimulate NK activity and cytokine production by NKT cells and exhibit potent antitumor activity in vivo (Kawano et al., 1997, Science 278: 1626-9; Kawano et al. 1998, supra; Kitamura et al. 1999, supra).

In addition to α-GalCer, other glycosylceramides having α-anomeric conformation of sugar moiety and 3,4-hydroxyl groups of the phytosphingosine (such as α-glucosylceramide [α-GlcCer], Galα1-6Galα1-1′Cer, Galα1-6Glcα1-1′Cer, Galα1-2Galα1-1′Cer, and Galβ1-3Galα1-1′Cer) have been demonstrated to stimulate proliferation of NKT cells in mice, although with lower efficiency (Kawano et al., Science, 278: 1626-1629, 1997, supra). By testing a panel of α-GalCer analogs for reactivity with mouse NKT cell hybridomas, Brossay et al. (J. Immunol., 161: 5124-5128, 1998) determined that nearly complete truncation of the α-GalCer acyl chain from 24 to 2 carbons does not significantly affect the mouse NKT cell response to glycolipid.

It has been also demonstrated that in vivo administration of α-GalCer not only causes the activation of NKT cells to induce a strong NK activity and cytokine production (e.g., IL-4, IL-12 and IFN-γ), but also induces the activation of immunoregulatory cells involved in acquired immunity (Nishimura et al., 2000, Int. Immunol., 12: 987-994). Specifically, in addition to the activation of macrophages and NKT cells, it was shown that in vivo administration of α-GalCer resulted in the induction of the early activation marker CD69 on CD4+ T cells, CD8+ T cells, and B cells (Burdin et al., 1999, Eur. J. Immunol. 29: 2014; Singh et al., 1999, J. Immunol. 163: 2373; Kitamura et al., 2000, Cell. Immunol. 199:37; Schofield et al., 1999, Science 283: 225; Eberl et al., 2000, J. Immunol., 165:4305-4311). These studies open the possibility that α-GalCer as well as other glycolipids may play an equally important role in bridging not only innate immunity mediated by NKT cells, but also adaptive immunity mediated by B cells, T helper (Th) cells and T cytotoxic (Tc) cells.

The demonstration that in vivo engagement of NKT cells by their glycolipid ligand α-GalCer rapidly induces a cascade of cellular activation that involves elements common to innate and adaptive immunity as well as the generation of tumor-specific cytotoxic T cells (Nishimura et al., 2000, supra) suggests that glycolipid administration may generally affect not only the speed and strength but also the type of subsequent immune responses, in particular, those directed against tumor cells. Indeed, Kabayashi et al. (1995, Oncol. Res., 7: 529-534) discovered that a synthetic form of α-GalCer (KRN 7000) had stronger antimetastatic activities in B16-bearing mice than biological response modifiers such as OK432 and Lentinan and a chemotherapeutic agent Mitomycin C. KRN 7000 was also shown to induce a pronounced tumor-specific immunity in mice with liver metastasis of murine T-lymphoma EL-4 cells (Nakagawa et al., Oncol. Res., 10: 561-568, 1998) or Colon26 cells (Nakagawa et al., Cancer Res., 58: 1202-1207, 1998). Furthermore, the administration of α-GalCer to mice was found to inhibit the development of hepatic metastasis of primary melanomas (Kawano et al., 1998, Proc. Natl. Acad. Sci. USA, 95: 5690-5693).

The present inventors and co-workers have recently demonstrated that the administration of α-GalCer to mice resulted rapidly in strong anti-malaria activity, inhibiting the development of intra-hepatocytic stages of the rodent malaria parasites, P. yoeli and P. berghei (Gonzalez-Aseguinolaza et al., 2000, Proc. Natl. Acad. Sci. USA, 97: 8461-8466). α-GalCer was unable to inhibit parasite development in the liver of mice lacking either IFN-γ or the IFN-γ receptor, indicating that the anti-malaria activity of the glycolipid is primarily mediated by IFN-γ.

Importantly, in addition to its ability to stimulate immune responses, it has been demonstrated that α-GalCer, independently of its dosage, does not induce toxicity in rodents and monkeys (Nakagawa et al., 1998, Cancer Res., 58: 1202-1207). Moreover, although a recent study showed the transient elevation of liver enzyme activities immediately after α-GalCer treatment in mice, suggesting a minor liver injury (Osman et al., 2000, Eur. J. Immunol., 39: 1919-1928), human trials are currently being conducted using α-GalCer to treat cancer patients without a notable complication (Giaccone et al., 2000, Abstract. Proc. Amer. Soc. Clin. Oncol., 19: 477a). See also Shimosaka et al. Cell Therapy: Filling the gap between basic science and clinical trials, First Int'l Workshop 2001, abstract pp. 21-22.

However, most mammals, including humans, have abundant amount of α-galactosidase, an enzyme which digests α-GalCer by catalyzing the degradation of α-D-galactoside bonds. As a result, α-GalCer has a short half-life, and therefore its in vivo therapeutic effect may be reduced.

Accordingly, there remains a strong need to identify and synthesize new C-glycolipids with improved stability and improved therapeutic efficacy over existing ones.

Taken together, there is a great need in the art to develop new adjuvants that would combine low in vivo toxicity, high in vivo stability and cost-efficient synthetic production with the ability to efficiently enhance and/or prolong the antigen-specific immune responses. The present invention addresses these and other needs in the art by providing novel synthetic C-glycolipids (and methods of making them) and demonstrates that these compounds have advantageous in vivo stability and immunostimulating properties and can be therefore used both directly and as adjuvants for augmenting immune responses in a mammal, notably a human, and can therefore improve prophylactic and/or therapeutic vaccines for the treatment of various infections, cancers and autoimmune diseases.

### OBJECTS OF THE INVENTION

It is an object of the invention to provide novel synthetic C-glycolipids and methods of making them, said novel synthetic C-glycolipids have advantageous stability and immunostimulating properties in vivo. It is also an object of the invention to use these novel compounds both directly and as immune adjuvants for treating cancers, infectious diseases and autoimmune diseases.

### SUMMARY OF THE INVENTION

This invention is directed to novel C-glycolipid compounds.

In a first embodiment, the invention provides novel C-glycolipid compounds represented by the general formula (I): 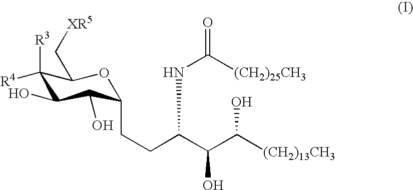
wherein X is O or NH;
R³ is OH or a monosaccharide and R⁴ is hydrogen, or R3 is hydrogen and R⁴ is OH or a monosaccharide;
R⁵ is hydrogen or a monosaccharide;
   and pharmaceutically acceptable salts or esters thereof.

The monosaccharide group(s) may be attached to the R³, R⁴ or R⁵ structure, to form a glycosyl bond. Typically, the monosaccharide is attached to the R³, R⁴ or R⁵ position at the oxygen attached to the C-1 carbon of the monosaccharide, forming the standard glycoside linkage.

A preferred compound of formula (I) is described by formula (I-a): 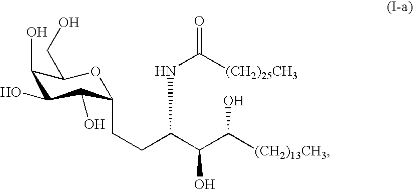
which is also referred to herein as GCM11i.

In a second embodiment, this invention provides novel C-glycolipid compounds represented by the general formula (II): 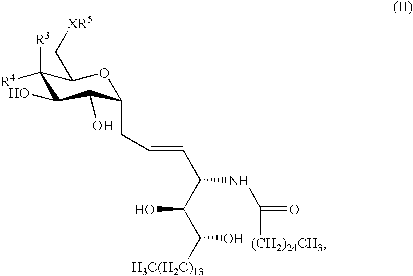
wherein X is O or NH;
R³ is OH or a monosaccharide and R⁴ is hydrogen, or R³ is hydrogen and R⁴ is OH or a monosaccharide;
R⁵ is hydrogen or a monosaccharide;
   and pharmaceutically acceptable salts or esters thereof.

The monosaccharide group(s) may be attached to the R³, R⁴ or R⁵ structure, to form a glycosyl bond. Typically, the monosaccharide is attached to the R³, R⁴ or R⁵ position at the oxygen attached to the C-1 carbon of the monosaccharide, forming the standard glycoside linkage.

A preferred compound of formula (II) is described by formula (II-a): 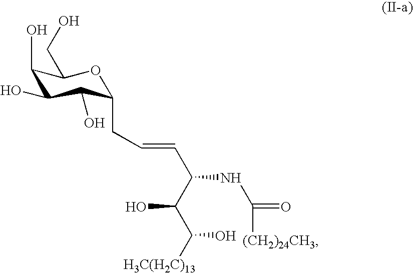
which is also referred to herein as GCK75(a).

In a third embodiment, the invention provides novel C-glycolipid compounds represented by the general formula (III): 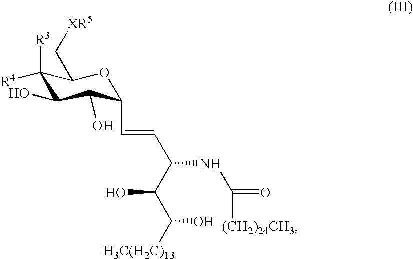
wherein X is O or NH;
R³ is OH or a monosaccharide and R⁴ is hydrogen, or R³ is hydrogen and R⁴ is OH or a monosaccharide;
R⁵ is hydrogen or a monosaccharide;
   and pharmaceutically acceptable salts or esters thereof.

The monosaccharide group(s) may be attached to the R³, R⁴ or R⁵ structure, to form a glycosyl bond. Typically, the monosaccharide is attached to the R³, R⁴ or R⁵ position at the oxygen attached to the C-1 carbon of the monosaccharide, forming the standard glycoside linkage.

Preferred compounds of formula (III) are described by formulas (III-a)(cis) and (III-a)(trans): 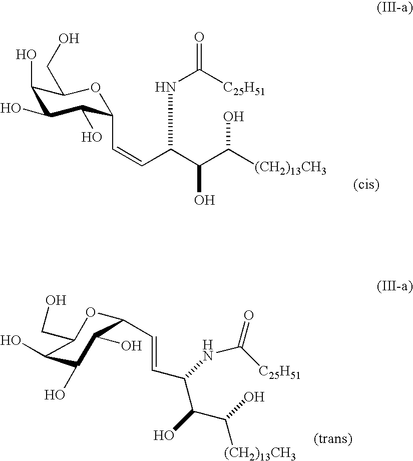
which are also referred to herein as GCK75(b).

In a another embodiment, the invention provides novel C-glycolipid compounds represented by the general formula (4): 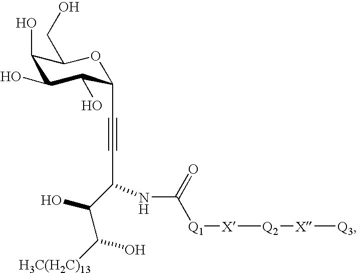
wherein Q¹ is optionally present and is a C₁₋₁₀ straight or branched chain alkylene, alkenylene, or alkynylene;
X′ is optionally present and is O, S or NR⁸;
Q² is optionally present and is a C₁₋₁₀ straight or branched chain alkylene, alkenylene or alkynylene;
X″ is optionally present and is O, S or NR⁸;
Q³ is a straight or branched chain C₁₋₁₀ alkyl, alkenyl or alkynyl, or is hydrogen,
wherein each Q¹, Q² or Q³ is optionally substituted with hydroxyl, halogen, cyano, nitro, SO₂, NHR⁸, or C(═O)—R⁹; and wherein
   R⁸ is hydrogen, C₁₋₅ alkyl, C₁₋₅ alkoxy, halogen, cyano, nitro, SO₂ or C(═O)—R⁹;
   R⁹ is hydrogen, C₁₋₅ alkyl, C₁₋₅ alkoxy or NHR¹⁰;
   R¹⁰ is hydrogen, C₁₋₅ alkyl or C₁₋₅ alkoxy;
      and pharmaceutically acceptable salts or esters thereof.

The invention is also directed to prodrugs and pharmaceutically acceptable salts of the compounds described, and to pharmaceutical compositions suitable for different routes of drug administration comprising a therapeutically effective amount of the described compounds of the invention admixed with a pharmaceutically acceptable carrier or excipient.

In conjunction with the novel C-glycolipid compounds and pharmaceutical compositions, the present invention provides methods of using these compounds and compositions both directly and as immune adjuvants to treat cancer, infections and autoimmune diseases. In a specific embodiment, the invention provides a method of using the compounds and compositions of the invention as immune adjuvants to augment an immunogenicity of an antigen in a mammal. In another specific embodiment, the invention provides a method of inducing the production of Th1 type cytokines, such as IFN-γ, in a mammal in need thereof, by administering to the mammal a therapeutically effective amount of the compounds and compositions of the invention. In yet another specific embodiment, the invention provides a method for treating a malarial infection using compounds and compositions of the invention. In preferred embodiments, the mammal is a human.

The invention also provides two novel synthesis methods which can be used to produce the compounds of the invention and other C-glycolipids.

In the first novel synthesis method, a compound of formula A: 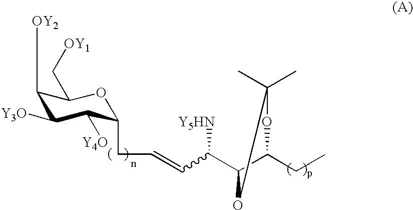
is formed by reacting 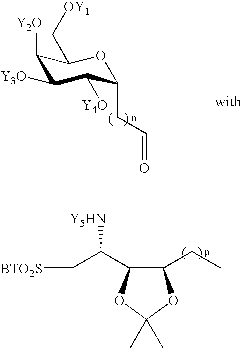
and a heterocyclic sulfone (see, e.g., P. R. Blakemore, J. Chem. Soc. Perkin I, 2002, 2563-2585);
   wherein
Y₁, Y₂, Y₃, and Y₄ are each independently protecting groups for sugar;
Y₅ is a protecting group for nitrogen;
n is 1 or 0; and
p is an integer from 1-100, preferably from 10-20, and most preferably 13.

Non-limiting examples of Y₁, Y₂, Y₃, and Y₄ include Ac (acetyl), Bn (benzyl), Bz (benzoate), PMB (para methoxybenzyl), TBDMS (tertiarybutyldimethylsilyl), TBDPS (tertiarybutyldiphenylsilyl), or connecting the oxygens of C4 and C6 with benzylidene or paramethoxybenzylidene (these add an additional ring). Preferably, Y₁, Y₂, Y₃, and Y₄ are each independently either Ac or Bn.

Non-limiting examples of Y₅ include CBZ (carbobenzyloxy), t-Boc (t-Butoxycarbonyl), FMOC (fluorenylmethyleneoxycarbonyl), and Phth (phthaloyl). Preferably Y₅ is either CBZ or t-Boc.

In the second novel synthesis method, a compound of formula (B) 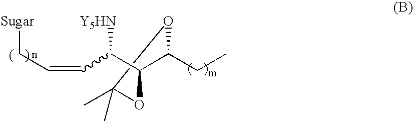
is formed by reacting 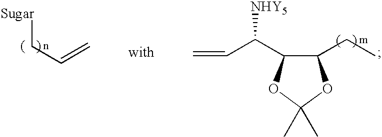
wherein
the sugar moiety can be protected or un-protected;
n is an integer from 0 to 20,
m is an integer from 1-100; and
Y₅ is a protecting group for nitrogen.

Preferably, the sugar is protected and selected from the group consisting of galactose, glucose, glucosamine, mannose, galactosamine, fucose, and rhamnose;
n is 1 or 0; and m is from an integer from 0-20, more preferably m is 13.

In a preferred embodiment of this method, a compound of formula (B-1) 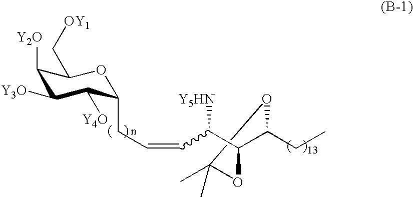
is formed by reacting 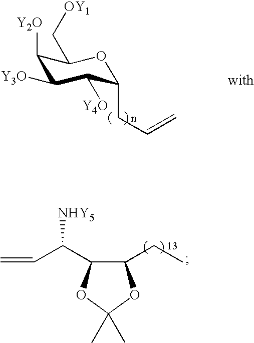
wherein
Y₁, Y₂, Y₃, and Y₄ are each independently protecting groups for sugar;
Y₅ is a protecting group for nitrogen; and
n is 0 or 1.

Non-limiting examples of Y₁, Y₂, Y₃, and Y₄ include Ac (acetyl), Bn (benzyl), Bz (benzoate), PMB (para methoxybenzyl), TBDMS (tertiarybutyldimethylsilyl), TBDPS (tertiarybutyldiphenylsilyl), or connecting the oxygens of C4 and C6 with benzylidene or paramethoxybenzylidene (these add an additional ring). Preferably, Y₁, Y₂, Y₃, and Y₄ are each independently either Ac or Bn.

Non-limiting examples of Y₅ include CBZ, t-Boc, FMOC (fluorenylmethyleneoxycarbonyl), and Phth (phthaloyl). Preferably Y₅ is either CBZ or t-Boc.

Compounds of formula (A), (B) and (B-1) are intermediates for making compounds of formula (C): 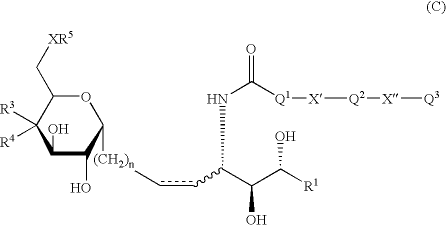
wherein X is O or NH;
n is 1 or 0;
R¹ is selected from the group consisting of —(CH₂)₁₁CH₃, —(CH₂)₁₂CH₃, —(CH₂)₁₃CH₃, —(CH₂)₉CH(CH₃)₂, —(CH₂)₁₀CH(CH₃)₂, —(CH₂)₁₁CH(CH₃)₂ and —(CH₂)₁₁CH(CH₃)—C₂H₅;
R³ is OH or a monosaccharide and R⁴ is hydrogen, or R³ is hydrogen and R⁴ is OH or a monosaccharide;
R⁵ is hydrogen or a monosaccharide;
Q¹ is optionally present and is a C₁₋₁₀ straight or branched chain alkylene, alkenylene, or alkynylene;
X′ is optionally present and is O, S or NR⁸;
Q² is optionally present and is a C₁₋₁₀ straight or branched chain alkylene, alkenylene or alkynylene;
X″ is optionally present and is O, S or NR⁸;
Q³ is a straight or branched chain C₁₋₁₀ alkyl, alkenyl or alkynyl, or is hydrogen,
wherein each Q¹, Q² or Q³ is optionally substituted with hydroxyl, halogen, cyano, nitro, SO₂, NHR⁸, or C(═O)—R⁹; and wherein
   R⁸ is hydrogen, C₁₋₅ alkyl, C₁₋₅ alkoxy, halogen, cyano, nitro, SO₂ or C(═O)—R⁹;
   R⁹ is hydrogen, C₁₋₅ alkyl, C₁₋₅ alkoxy or NHR¹⁰;
   R¹⁰ is hydrogen, C₁₋₅ alkyl or C₁₋₅ alkoxy;
      and pharmaceutically acceptable salts or esters thereof.

The monosaccharide groups may be attached to the R³, R⁴ or R⁵ structure, to form a glycosyl bond. Typically, the monosaccharide is attached to the R³, R⁴ or R⁵ position at the oxygen attached to the C-1 carbon of the monosaccharide, forming the standard glycoside linkage.

### BRIEF DESCRIPTION OF THE DRAWINGS

FIG. 1 is a graph showing a time line of IFN-γ concentrations (as determined by ELISA at 2, 6, 12, and 24 hours post-injection) in the sera of BALB/c mice injected i.v. with 1 μg of α-GalCer (KRN), α-C-GalCer (CRONY), GCM11i (compound “i”), GCK75(a) (compound “a”), GCK75(b) (compound “b”), or nothing. α-GalCer (KRN) or α-C-GalCer (CRONY) administration induces IFN-γ production in the sera, peaking at 12 or 24 hours post-injection, respectively. GCM11i induces a peak IFN-γ response at 6 hours post-injection, whereas GCK75(b) induces the peak response more than 24 hours post-injection.

FIG. 2 is a bar graph showing the amounts of parasite-specific 18S rRNA (as determined by quantitative real-time RT-PCR) in the livers of BALB/c mice injected i.v. with 1 μg of GCM11i (compound “i”), GCK75(b) (compound “b”), α-C-GalCer (CRONY) (positive control), or nothing (negative control) two days before challenge with live P. yoelii sporozoites. Both GCM11i and GCK75(b) display a level of anti-malarial activity comparable to that of α-C-GalCer (CRONY).

### DETAILED DESCRIPTION OF THE INVENTION

Definitions

The term “monosaccharide” means a sugar molecule having a chain of 3-10 carbon atoms in the form of an aldehyde (aldose) or ketone (ketose). Suitable monosaccharides contemplated for use in the invention include both naturally occurring and synthetic monosaccharides. Sample monosaccharides include trioses, such as glycerose and dihydroxyacetone; textroses such as erythrose and erythrulose; pentoses such as xylose, arabinose, ribose, xylulose ribulose; methyl pentoses (6-deoxyhexoses), such as rhamnose and fucose; hexoses, such as glucose, mannose, galactose, fructose and sorbose; and heptoses, such as glucoheptose, galamannoheptose, sedoheptulose and mannoheptulose. Preferred monosaccharides are hexoses.

An “effective amount” of the compound for treating a disease, e.g., a cancer, an infectious disease or an autoimmune disease, is an amount that results in measurable amelioration of at least one symptom or parameter of the disease in mammals, including humans.

The term “prodrug” as used herein refers to any compound that may have less intrinsic activity than the active compound or “drug” but when administered to a biological system generates the active compound or “drug” substance either as a result of spontaneous chemical reaction or by enzyme catalyzed or metabolic reaction.

As used herein, the term “pharmaceutically acceptable salts, esters, amides, and prodrugs” refer to those salts (e.g., carboxylate salts, amino acid addition salts), esters, amides, and prodrugs of the compounds of the present invention which are, within the scope of sound medical judgment, suitable for use in contact with the tissues of patients without undue toxicity, irritation, allergic response, and the like, commensurate with a reasonable benefit/risk ratio, and effective for their intended use, as well as the zwitterionic forms, where possible, of the compounds of the invention.

The term “treat” is used herein to mean to relieve or alleviate at least one symptom of a disease in a subject and includes any benefits obtained or derived from the administration of the described compounds. Within the meaning of the present invention, the term “treat” includes prophylactic or therapeutic administration of compounds of the invention and may also mean to prolong the prepatency, i.e., the period between infection and clinical manifestation of a disease. Preferably, the disease is either infectious disease (e.g., viral, bacterial, parasitic, or fungal) or malignancy (e.g., solid or blood tumors such as sarcomas, carcinomas, gliomas, blastomas, pancreatic cancer, breast cancer, ovarian cancer, prostate cancer, lymphoma, leukemia, melanoma, etc.) or an autoimmune disease.

The term “therapeutically effective” applied to dose or amount refers to that quantity of a compound or pharmaceutical composition or vaccine that is sufficient to result in a desired activity upon administration to a mammal in need thereof. As used herein with respect to adjuvant and/or immunostimulating compositions or vaccines, the term “therapeutically effective amount/dose” is used interchangeably with the term “immunogenically effective amount/dose” and refers to the amount/dose of a compound or pharmaceutical composition or vaccine that is sufficient to produce an effective immune response upon administration to a mammal.

The terms “pharmaceutically acceptable” and “physiologically acceptable” are used interchangeably, and as used in connection with compositions of the invention refer to molecular entities and other ingredients of such compositions that are physiologically tolerable and do not typically produce untoward reactions when administered to a human. Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans.

The terms “adjuvant” and “immunoadjuvant” are used interchangeably in the present invention and refer to a compound or mixture that may be non-immunogenic when administered to a host alone, but that augments the host's immune response to another antigen when administered conjointly with that antigen.

As used herein, the term “augment the immune response” means enhancing or extending the duration of the immune response, or both. When referred to a property of an agent (e.g., adjuvant), the term “[able to] augment the immunogenicity” refers to the ability to enhance the immunogenicity of an antigen or the ability to extend the duration of the immune response to an antigen, or both.

The phrase “enhance immune response” within the meaning of the present invention refers to the property or process of increasing the scale and/or efficiency of immunoreactivity to a given antigen, said immunoreactivity being either humoral or cellular immunity, or both. An immune response is believed to be enhanced, if any measurable parameter of antigen-specific immunoreactivity (e.g., antibody titer, T cell production) is increased at least two-fold, preferably ten-fold, most preferably thirty-fold.

The term “carrier” applied to pharmaceutical compositions of the invention refers to a diluent, excipient, or vehicle with which a compound of the invention is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution, saline solutions, and aqueous dextrose and glycerol solutions are preferably used as carriers, particularly for injectable solutions. Suitable pharmaceutical carriers are described in “Remington's Pharmaceutical Sciences” by E. W. Martin, 18th Edition.

As used herein, the term “immunogenic” means that an agent is capable of eliciting a humoral or cellular immune response, and preferably both. An immunogenic entity is also antigenic. An immunogenic composition is a composition that elicits a humoral or cellular immune response, or both, when administered to an animal having an immune system.

The term “vaccine” refers to a composition (e.g., protein or vector such as, e.g., an adenoviral vector, Sindbis virus vector, or pox virus vector) that can be used to elicit protective immunity in a recipient. It should be noted that to be effective, a vaccine of the invention can elicit immunity in a portion of the immunized population, as some individuals may fail to mount a robust or protective immune response, or, in some cases, any immune response. This inability may stem from the individual's genetic background or because of an immunodeficiency condition (either acquired or congenital) or immunosuppression (e.g., due to treatment with chemotherapy or use of immunosuppressive drugs, e.g., to prevent organ rejection or suppress an autoimmune condition). Vaccine efficacy can be established in animal models.

The term “subject” as used herein refers to an animal having an immune system, preferably a mammal (e.g., rodent such as mouse). In particular, the term refers to humans.

The term “about” or “approximately” usually means within 20%, more preferably within 10%, and most preferably still within 5% of a given value or range. Alternatively, especially in biological systems (e.g., when measuring an immune response), the term “about” means within about a log (i.e., an order of magnitude) preferably within a factor of two of a given value.

Therapeutic Uses

In one embodiment, the compounds of the invention are useful for the treatment of cancer, e.g., as immune adjuvants in combination with cancer-specific antigens and/or directly as anti-tumor agents for inhibiting the growth of tumors, and for treatment of cell proliferative disorders. The compounds of the invention may be used alone, or in combination with chemotherapy or radiotherapy.

More specifically, the compounds of the invention are useful in the treatment of a variety of cancers including, but not limited to carcinoma such as bladder, breast, colon, kidney, liver, lung, including small cell lung cancer, non-small cell lung cancer, esophagus, gall bladder, ovary, pancreas, testicular, stomach, renal, liver, cervix, thyroid, prostate, and skin, including squamous cell carcinoma; hematopoietic tumors of lymphoid lineage, including leukemia, acute lymphocitic leukemia, acute lymphoblastic leukemia, B cell lymphoma, T cell lymphoma, Hodgkin's lymphoma, non-Hodgkin's lymphoma, hairy cell lymphoma and Burkett's lymphoma; hematopoietic tumors of myeloid lineage, including acute and chronic myelogenous leukemias, myelodysplastic syndrome and promyelocytic leukemia; tumors of mesenchymal origin, including fibrosarcoma and rhabdomyosarcoma; tumors of the central and peripheral nervous system, including astrocytoma, neuroblastoma, glioma and schwannomas; other tumors, including melanoma, seminoma, teratocarcinoma, osteosarcoma, xenoderoma pigmentosum, keratoctanthoma, thyroid follicular cancer and Kaposi's sarcoma.

Cell proliferative disorders for which the compounds are useful include benign prostate hyperplasia, familial adenomatosis polyposis, neuro fibromatosis, psoriasis, vascular smooth cell proliferation associated with atherosclerosis, pulmonary fibrosis, arthritis glomerulonephritis and post-surgical stenosis and restenosis.

In another embodiment, the compounds of the invention are also useful both directly and as immune adjuvants for treating and/or preventing infectious diseases, including parasitic, fungal, yeast, bacterial, mycoplasmal and viral diseases (where a particular class of cells can be identified as harboring the infective entity).

For example, the compounds may be useful in treating and/or preventing infections from a human papilloma virus, a herpes virus such as herpes simplex or herpes zoster, a retrovirus such as human immunodeficiency virus (HIV) 1 or 2, a hepatitis virus (hepatitis A virus (HAV)), hepatitis B virus (HBV) non-A, blood borne (hepatitis C) and other enterically transmitted hepatitis (hepatitis E), and HBV associated delta agent (hepatitis D)), influenza virus, rhinovirus, respiratory syncytial virus, cytomegalovirus, adenovirus, Mycoplasma pneumoniae, a bacterium of the genus Salmonella, Staphylococcus, Streptococcus, Enterococcus, Clostridium, Escherichia, Klebsiella, Vibrio, Mycobacterium, amoeba, a malarial parasite, Trypanosoma cruzi, helminth infections, such as nematodes (round worms) (Trichuriasis, Enterobiasis, Ascariasis, Hookworm, Strongyloidiasis, Trichinosis, filariasis); trematodes (flukes) (Schistosomiasis, Clonorchiasis), cestodes (tape worms) (Echinococcosis, Taeniasis saginata, Cysticercosis); visceral worms, visceral larva migrans (e.g., Toxocara), eosinophilic gastroenteritis (e.g., Anisaki spp., Phocanema ssp.), cutaneous larva migrans (Ancylostona braziliense, Ancylostoma caninum).

In another embodiment, the compounds of the invention are useful both directly and as immune adjuvants for treating and/or preventing autoimmune diseases, such as rheumatoid arthritis, psoriatic arthritis, multiple sclerosis, systemic lupus erythematosus, myasthenia gravis, juvenile onset diabetes, glomerulonephritis, autoimmune thyroiditis, Behcet's disease, and other disorders such as Crohn's disease, ulcerative colitis, bullous pemphigoid, sarcoidosis, psoriasis, ichthyosis, Graves ophthalmopathy and asthma.

The subjects to which the present invention is applicable may be any mammalian or vertebrate species, which include, but are not limited to, cows, horses, sheep, pigs, fowl (e.g., chickens), goats, cats, dogs, hamsters, mice, rats, monkeys, rabbits, chimpanzees, and humans. In a preferred embodiment, the subject is a human.

Modes of Administration

Modes of administration of compounds and compositions of the invention include oral and enteral, intravenous, intramuscular, subcutaneous, transdermal, transmucosal (including rectal and buccal), and by inhalation routes. Preferably, an oral or transdermal route is used (i.e., via solid or liquid oral formulations, or skin patches, respectively). In some cases, the compounds can be pulsed with syngeneic dendritic cells, followed by transferring intravenously into patients.

Pharmaceutical Compositions

Solid dosage forms for oral administration of compounds and compositions of the invention include capsules, tablets, pills, powders, granules, and suppositories. In such solid dosage forms, the active compound of the invention can be admixed with at least one inert customary excipient (or carrier) such as sodium citrate or dicalcium phosphate; or (a) fillers or extenders, as for example, starches, lactose, sucrose, glucose, mannitol, and silicic acid; (b) binders, as for example, carboxymethylcellulose, alignates, gelatin, polyvinylpyrrolidone, sucrose, and acacia; (c) humectants, as for example, glycerol; (d) disintegrating agents, as for example, agar-agar, calcium carbonate, potato or tapioca starch, alginic acid, certain complex silicates, and sodium carbonate; (e) solution retarders, as for example paraffin; (f) absorption accelerators, as for example, quaternary ammonium compounds; (g) wetting agents, as for example, cetyl alcohol, and glycerol monostearate; (h) adsorbents, as for example, kaolin and bentonite; and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents. Such solid compositions or solid compositions that are similar to those described can be employed as fillers in soft- and hard-filled gelatin capsules using excipients such as lactose or milk, sugar as well as high molecular weight polyethyleneglycols, and the like.

Solid dosage forms such as tablets, dragées, capsules, pills, and granules can be prepared with coatings and shells, such as enteric coatings or other suitable coatings or shells. Several such coatings and/or shells are well known in the art, and can contain opacifying agents, and can also be of such composition that they release the active compound or compounds in a certain part of the intestinal tract in a delayed manner. Examples of embedding compositions which can be used are polymeric substances and waxes. The active compounds can also be used in microencapsulated form, if appropriate, with one or more of the above-mentioned excipients.

Liquid dosage forms for oral administration include pharmaceutically acceptable emulsions, solutions, suspensions, syrups, and elixirs. In addition to the active compounds, the liquid dosage forms can contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan or mixtures of these substances, and the like. If desired, the composition can also include adjuvants, such as wetting agents, emulsifying and suspending agents, sweetening, flavoring and/or perfuming agents.

The composition may include a carrier, as defined herein. Suitable carriers include macromolecules which are soluble in the circulatory system and which are physiologically acceptable, as defined herein. The carrier preferably is relatively stable in the circulatory system with an acceptable plasma half life for clearance. Such macromolecules include but are not limited to Soya lecithin, oleic acid and sorbitan trioleate, with sorbitan trioleate preferred.

Suspensions, in addition to the active compounds, can contain suspending agents, such as, ethoxylated isostearyl alcohols, polyoxyethylene sorbitol and sorbitan esters, microcrystalline cellulose, aluminum metahydroxide, bentonite, agar-agar, tragacanth, and the like. Mixtures of suspending agents can be used if desired.

Compositions for rectal administrations are preferably suppositories which can be prepared by mixing the compounds of the present invention with suitable nonirritating excipients or carriers such as cocoa butter, polyethyleneglycol, or a suppository wax which are solid at ordinary temperatures but liquid at body temperature and therefore, melt in the rectum or vaginal cavity and release the active component.

Compositions suitable for parenteral injection can comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

Dosage forms for topical administration of a compound of the invention include ointments, powders, sprays and inhalants. The active component can be admixed under suitable conditions (e.g., sterile conditions) with a physiologically acceptable carrier and any preservatives, buffers, or propellants as may be required. Ophthalmic formulations, eye ointments, powders, and solutions are also contemplated as being within the scope of this invention.

Effective Dosages

An effective amount for treating the diseases can easily be determined by empirical methods known to those skilled in the art, such as by establishing a matrix of dosages and frequencies of administration and comparing a group of experimental units or subjects to each point in the matrix. The exact amount to be administered to a patient will vary depending on the particular disease, the state and severity of the disease, and the physical condition of the patient. A measurable amelioration of any symptom or parameter can be determined by a physician skilled in the art or reported by the patient to the physician. Clinically significant attenuation or amelioration means perceptible to the patient and/or to the physician.

It will also be understood that the specific dosage form and dose level for any particular patient will depend on a variety of factors including the activity of the specific compound employed; the age, body weight, general health, and sex of the individual being treated; the time and route of administration; the rate of excretion; other drugs which have previously been administered; and the severity of the particular disease undergoing therapy.

The amount of the agent to be administered can range from between about 0.01 to about 25 mg/kg/day, preferably from between about 0.1 to about 10 mg/kg/day and most preferably from between about 0.2 to about 5 mg/kg/day. It will be understood that the pharmaceutical compositions of the present invention need not in themselves contain the entire amount of the agent that is effective in treating the disorder, as such effective amounts can be reached by administration of a plurality of doses of such pharmaceutical compositions.

For example, the compounds of the invention can be formulated in capsules or tablets, each preferably containing 50-200 mg of the compounds of the invention, and are most preferably administered to a patient at a total daily dose of 50-400 mg, preferably 150-250 mg, and most preferably about 200 mg.

Toxicity and therapeutic efficacy compositions containing compounds of the invention can be determined by standard pharmaceutical procedures in experimental animals, e.g., by determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compositions that exhibit large therapeutic indices are preferred. While therapeutics that exhibit toxic side effects can be used (e.g., when treating severe forms of cancer or life-threatening infections), care should be taken to design a delivery system that targets such immunogenic compositions to the specific site (e.g., lymphoid tissue mediating an immune response, tumor or an organ supporting replication of the infectious agent) in order to minimize potential damage to other tissues and organs and, thereby, reduce side effects.

As specified above, data obtained from the animal studies can be used in formulating a range of dosage for use in humans. The therapeutically effective dosage of compounds of the present invention in humans lies preferably within a range of circulating concentrations that include the ED50 with little or no toxicity. The dosage can vary within this range depending upon the dosage form employed and the route of administration utilized. Ideally, a single dose should be used.

Novel Compounds of the Invention

In a first embodiment of the novel compound, the invention is directed to novel C-glycolipid compound of formula (I) 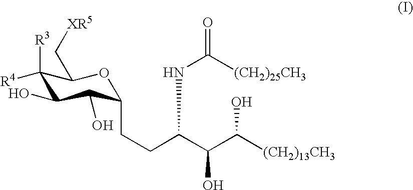
wherein X is O or NH;
R³ is OH or a monosaccharide and R⁴ is hydrogen, or R³ is hydrogen and R⁴ is OH or a monosaccharide;
R⁵ is hydrogen or a monosaccharide;
   and pharmaceutically acceptable salts or esters thereof.

The monosaccharide group(s) may be attached to the R³, R⁴ or R⁵ structure, to form a glycosyl bond. Typically, the monosaccharide is attached to the R³, R⁴ or R⁵ position at the oxygen attached to the C-1 carbon of the monosaccharide, forming the standard glycoside linkage.

A preferred compound of formula (I) is described by formula (I-a) 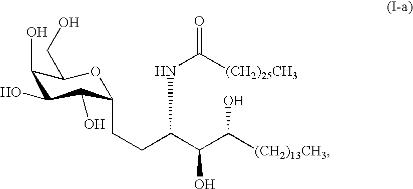
which is also known as GCM11i.

In a second embodiment of the novel compounds, this invention is directed to novel C-glycolipid compound of formula (II) 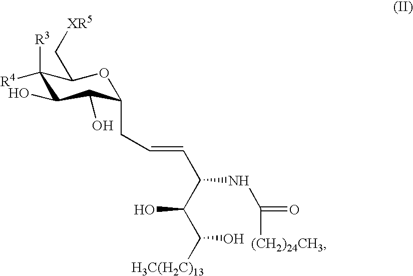
wherein X is O or NH;
R³ is OH or a monosaccharide and R⁴ is hydrogen, or R³ is hydrogen and R⁴ is OH or a monosaccharide;
R⁵ is hydrogen or a monosaccharide;
   and pharmaceutically acceptable salts or esters thereof.

The monosaccharide group(s) may be attached to the R³, R⁴ or R⁵ structure, to form a glycosyl bond. Typically, the monosaccharide is attached to the R³, R⁴ or R⁵ position at the oxygen attached to the C-1 carbon of the monosaccharide, forming the standard glycoside linkage.

A preferred compound of formula (II) is described by formula (II-a) is 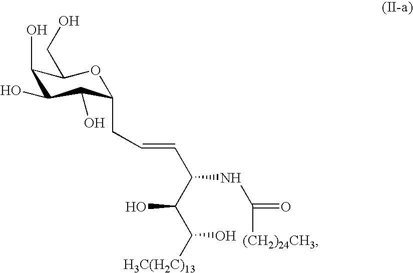
which is also known as GCK75(a).

In a third embodiment of the novel compounds, this invention is directed to novel C-glycolipid compound of formula (III) 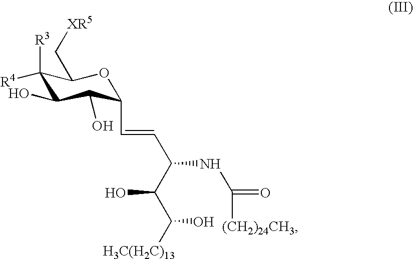
wherein X is O or NH;
R³ is OH or a monosaccharide and R⁴ is hydrogen, or R³ is hydrogen and R⁴ is OH or a monosaccharide;
R⁵ is hydrogen or a monosaccharide;
   and pharmaceutically acceptable salts or esters thereof.

The monosaccharide group(s) may be attached to the R³, R⁴ or R⁵ structure, to form a glycosyl bond. Typically, the monosaccharide is attached to the R³, R⁴ or R⁵ position at the oxygen attached to the C-1 carbon of the monosaccharide, forming the standard glycoside linkage.

Preferred compounds of formula (III) are described by formulas (III-a)(cis) and (III-a)(trans): 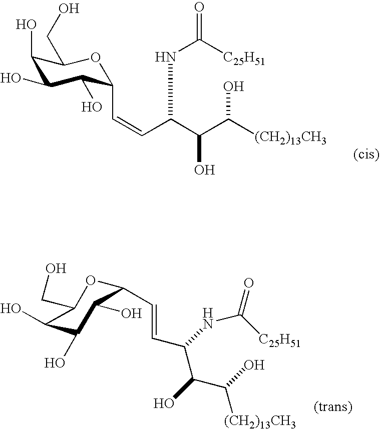
which are also known as GCK75(b).

In a another embodiment, the invention provides novel C-glycolipid compounds represented by the general formula (4): 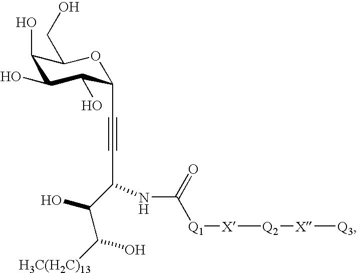
wherein Q¹ is optionally present and is a C₁₋₁₀ straight or branched chain alkylene, alkenylene, or alkynylene;
X′ is optionally present and is O, S or NR⁸;
Q² is optionally present and is a C₁₋₁₀ straight or branched chain alkylene, alkenylene or alkynylene;
X″ is optionally present and is O, S or NR⁸;
Q³ is a straight or branched chain C₁₋₁₀ alkyl, alkenyl or alkynyl, or is hydrogen,
wherein each Q¹, Q² or Q³ is optionally substituted with hydroxyl, halogen, cyano, nitro, SO₂, NHR⁸, or C(═O)—R⁹; and wherein
   R⁸ is hydrogen, C₁₋₅ alkyl, C₁₋₅ alkoxy, halogen, cyano, nitro, SO₂ or C(═O)—R⁹;
   R⁹ is hydrogen, C₁₋₅ alkyl, C₁₋₅ alkoxy or NHR¹⁰;
   R¹⁰ is hydrogen, C₁₋₅ alkyl or C₁₋₅ alkoxy;
      and pharmaceutically acceptable salts or esters thereof.
      Synthesis Method A

A compound of formula A: 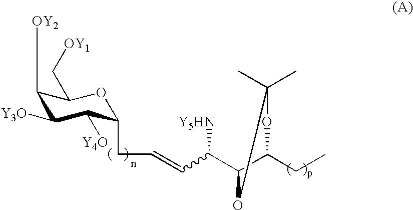
is formed using Julia-Kocienski olefination procedure by reacting 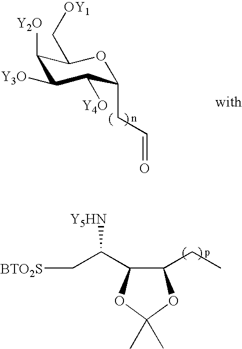
and a heterocyclic sulfone;
   wherein
Y₁, Y₂, Y₃, and Y₄ are each independently protecting groups for sugar;
Y₅ is a protecting group for nitrogen;
n is 1 or 0;
p is an integer from 1-100, preferably from 10-20, and most preferably 13.

Non-limiting examples of Y₁, Y₂, Y₃, and Y₄ include Ac (acetyl), Bn (benzyl), Bz (benzoate), PMB (para methoxybenzyl), TBDMS (tertiarybutyldimethylsilyl), TBDPS (tertiarybutyldiphenylsilyl), or connecting the oxygens of C4 and C6 with benzylidene or paramethoxybenzylidene (these add an additional ring). Preferably, Y₁, Y₂, Y₃, and Y₄ are each independently either Ac or Bn.

Non-limiting examples of Y₅ include CBZ, t-Boc, FMOC (fluorenylmethyleneoxycarbonyl), and Phth (phthaloyl). Preferably Y₅ is either CBZ or t-Boc.

The starting materials of this reaction can be prepared according to the methods described below or other methods known in the art.

Compound of Formula (A) can be used to synthesize compounds of formula (C) using methods described herein or methods known to one skilled in the art: 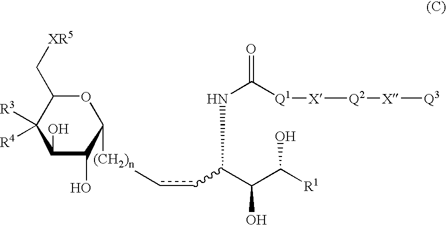
wherein X is O or NH;
n is 1 or 0;
R¹ is selected from the group consisting of —(CH₂)₁₁CH₃, —(CH₂)₁₂CH₃, —(CH₂)₁₃CH₃, —(CH₂)₉CH(CH 3)₂, —(CH₂)₁₀CH(CH₃)₂, —(CH₂)₁₁CH(CH₃)₂ and —(CH₂)₁₁CH(CH₃)—C₂H₅;
R³ is OH or a monosaccharide and R⁴ is hydrogen, or R is hydrogen and R⁴ is OH or a monosaccharide;
R⁵ is hydrogen or a monosaccharide;
Q¹ is optionally present and is a C₁₋₁₀ straight or branched chain alkylene, alkenylene, or alkynylene;
X′ is optionally present and is O, S or NR⁸;
Q² is optionally present and is a C₁₋₁₀ straight or branched chain alkylene, alkenylene or alkynylene;
X″ is optionally present and is O, S or NR⁸;
Q³ is a straight or branched chain C₁₋₁₀ alkyl, alkenyl or alkynyl, or is hydrogen,
wherein each Q¹, Q² or Q³ is optionally substituted with hydroxyl, halogen, cyano, nitro, SO₂, NHR⁸, or C(═O)—R⁹; and wherein
   R⁸ is hydrogen, C₁₋₅ alkyl, C₁₋₅ alkoxy, halogen, cyano, nitro, SO₂ or C(═O)—R⁹;
   R⁹ is hydrogen, C₁₋₅ alkyl, C₁₋₅ alkoxy or NHR¹⁰;
   R¹⁰ is hydrogen, C₁₋₅ alkyl or C₁₋₅ alkoxy;
      and pharmaceutically acceptable salts or esters thereof.

The monosaccharide groups may be attached to the R³, R⁴ or R⁵ structure, to form a glycosyl bond. Typically, the monosaccharide is attached to the R³, R⁴ or R⁵ position at the oxygen attached to the C-1 carbon of the monosaccharide, forming the standard glycoside linkage.

Exemplary compounds of Formula C include but not limited to: 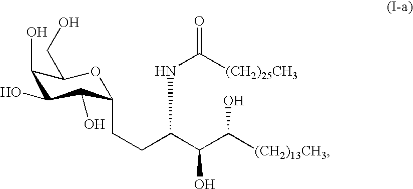
which is also known as GCM11i; 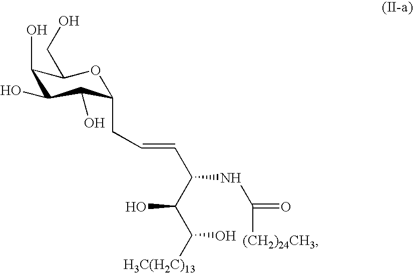
which is also known as GCK75(a); 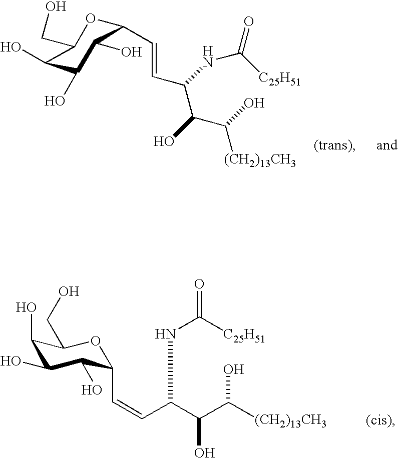
which are also known as GCK75(b); and 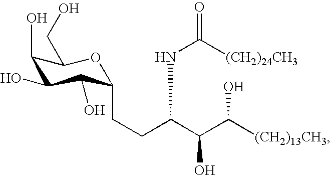
which is also known as CRONY 101.
Synthesis Method B

A compound of formula B 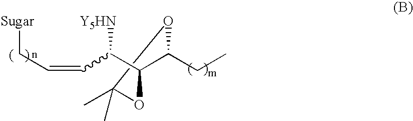
is formed using an olefin metathesis procedure by reacting 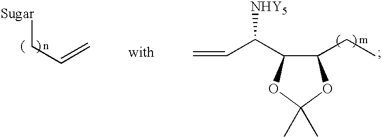
wherein
the sugar moiety can be protected or un-protected;
n is an integer from 0 to 20,
m is an integer from 1-100; and
Y₅ is a protecting group for nitrogen.

Preferably, the sugar is protected and selected from the group consisting of galactose, glucose, glucosamine, mannose, galactosamine, fucose, and rhamnose;
n is 1 or 0; m is an integer from 10-20, more preferably 13.

In a preferred embodiment of this method, a compound of formula (B-1) 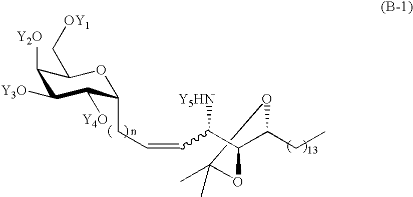
is formed using an olefin metathesis procedure by reacting 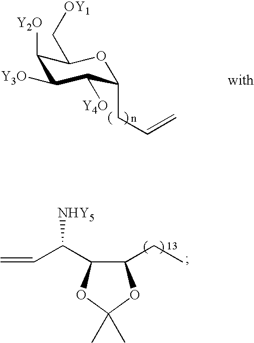
wherein
Y₁, Y₂, Y₃, and Y₄ are each independently protecting groups for sugar;
Y₅ is a protecting group for nitrogen; and
n is 0 or 1.

Non-limiting examples of Y₁, Y₂, Y₃, and Y₄ include Ac (acetyl), Bn (benzyl), Bz (benzoate), PMB (para methoxybenzyl), TBDMS (tertiarybutyldimethylsilyl), TBDPS (tertiarybutyldiphenylsilyl), or connecting the oxygens of C4 and C6 with benzylidene or paramethoxybenzylidene (these add an additional ring). Preferably, Y₁, Y₂, Y₃, and Y₄ are each independently either Ac or Bn.

Non-limiting examples of Y₅ include CBZ, t-Boc, FMOC (fluorenylmethyleneoxycarbonyl), and Phth (phthaloyl). Preferably Y₅ is either CBZ or t-Boc.

The starting materials of this reaction can be prepared according to the methods described below or other methods known in the art.

Compound of Formula (B) or (B-1) can be used to synthesize compounds of formula (C) using methods described herein or methods known to one skilled in the art: 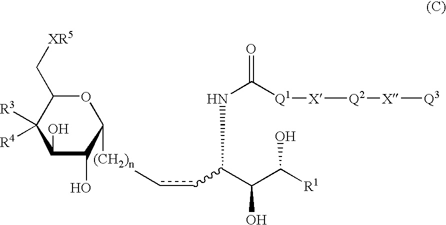
wherein X is O or NH;
n is 1 or 0;
R¹ is selected from the group consisting of —(CH₂)₁₁CH₃, —(CH₂)₁₂CH₃, —(CH₂)₁₃CH₃, —(CH₂)₉CH(CH₃)₂, —(CH₂)₁₀CH(CH₃)₂, —(CH₂)₁₁CH(CH₃)₂ and —(CH₂)₁₁CH(CH₃)—C₂H₅;
R³ is OH or a monosaccharide and R⁴ is hydrogen, or R³ is hydrogen and R⁴ is OH or a monosaccharide;
R⁵ is hydrogen or a monosaccharide;
Q¹ is optionally present and is a C₁₋₁₀ straight or branched chain alkylene, alkenylene, or alkynylene;
X′ is optionally present and is O, S or NR⁸;
Q² is optionally present and is a C₁₋₁₀ straight or branched chain alkylene, alkenylene or alkynylene;
X″ is optionally present and is O, S or NR⁸;
Q³ is a straight or branched chain C₁₋₁₀ alkyl, alkenyl or alkynyl, or is hydrogen,
wherein each Q¹, Q² or Q³ is optionally substituted with hydroxyl, halogen, cyano, nitro, SO₂, NHR⁸, or C(═O)—R⁹; and wherein
   R⁸ is hydrogen, C₁₋₅ alkyl, C₁₋₅ alkoxy, halogen, cyano, nitro, SO₂ or C(═O)—R⁹;
   R⁹ is hydrogen, C₁₋₅ alkyl, C₁₋₅ alkoxy or NHR¹⁰;
   R¹⁰ is hydrogen, C₁₋₅ alkyl or C₁₋₅ alkoxy;
      and pharmaceutically acceptable salts or esters thereof.

The monosaccharide groups may be attached to the R³, R⁴ or R⁵ structure, to form a glycosyl bond. Typically, the monosaccharide is attached to the R³, R⁴ or R⁵ position at the oxygen attached to the C-1 carbon of the monosaccharide, forming the standard glycoside linkage.

Exemplary compounds of Formula C include but not limited to: 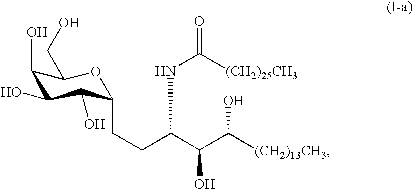
which is also known as GCMK11i; 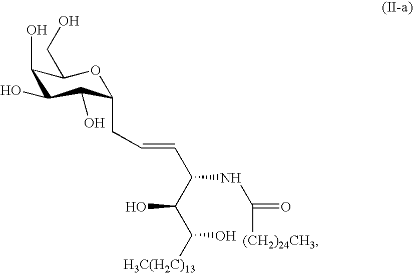
which is also known as GCK75(a); 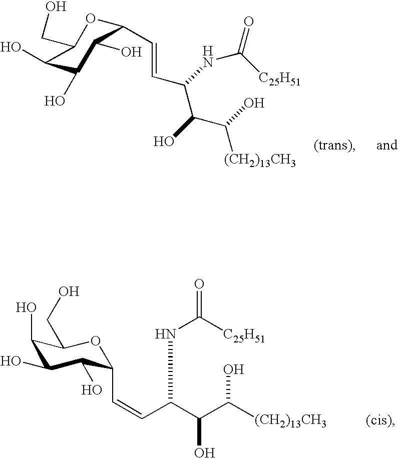
which are also known as GCK75(b); and 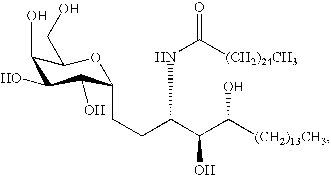
which is also known as CRONY 101.

The following Examples illustrate the invention without limiting its scope.

### EXAMPLES

The compounds of this invention and their preparation and the methods of their use can be understood further by the examples which illustrate some of the processes by which these compounds are prepared or used. Theses examples do not limit the invention. Variations of the invention, now known or further developed, are considered to fall within the scope of the present invention as hereinafter claimed.
Chemical Compounds and Chemical Synthesis

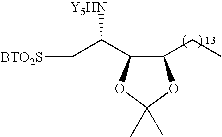

### Example A

### Preparation of Y₅: CBZ or tBoc

The reaction scheme was carried as follows: 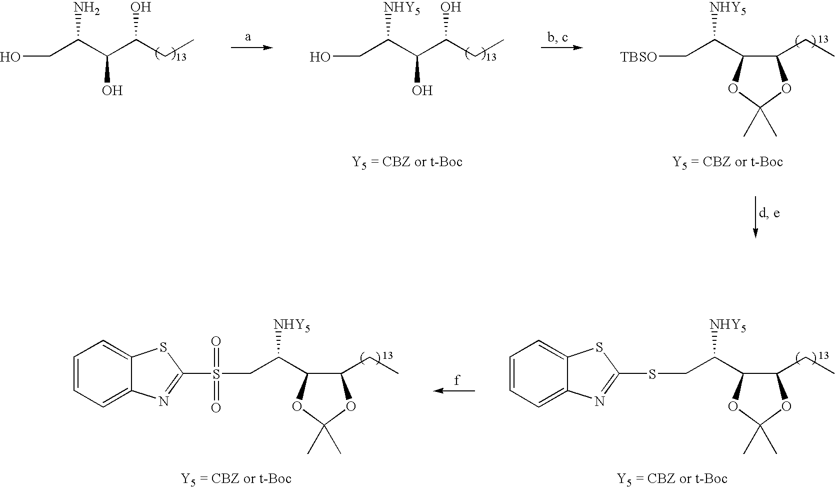
Conditions:

Step (a):
for benzyl carbamate: CBZCl (1.1 eq.), 1N NaHCO₃, 1,4-dioxane, ethyl acetate, rt (room temperature), overnight, the yield was 90%;
for t-butyl carbamate: 1N NaOH (1.5 eq.), (t-Boc)₂O (1.5 eq.), ethanol, water, rt, 1 h;

Step (b):
for benzyl carbamate: TBSCl (t-butyldimethylsilyl chloride) (1.2 eq.), Et₃N (1.1 eq.), 4-DMAP (4-dimethylaminopyridine) (0.05 eq.), DCM (dichloromethane), DMF (dimethylformamide), 0° C., 1 h, 96%;
for t-butyl carbamate: The overall yield for steps (a) and (b) was 93%.

Step (c):
for benzyl carbamate: 2,2-dimethoxypropane (5-10 eq.), PPTs (pyridinium p-toluenesulfonate) (0.07 eq.), DCM, rt, 2 h, the yield was 99%;
for t-butyl carbamate: the reaction product was directly used for next step without purification;

Step (d):
for benzyl carbamate: TBAF (tetrabutylammonium fluoride) (2.0 eq.), HOAc (trace), THF (tetrahydrofuran), rt, 4 h, the yield was 99%;
for t-butyl carbamate: TBAF (1.2 eq.), THF, 0° C.-rt, 3 h. The overall yield for steps (c) and (d) was 94%.

Step (e):
for benzyl carbamate: Ph₃P (1.1 eq.), BTSH (1.1 eq.), DiPAD (diisopropyl azodicarboxylate) (1.1 eq.), THF, rt, 3 h, the yield was 93%;
for t-butyl carbamate: the yield was 97%;

Step (f):

NaHCO₃ (5.0 eq.), MCPBA (meta chloroperbenzoic acid) (2.5 eq.), DCM, rt, overnight, 98%.

### Example B

### Preparation of Sugar Aldehydes

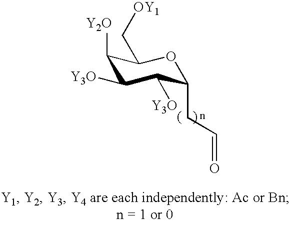

The reaction scheme was carried as follows: 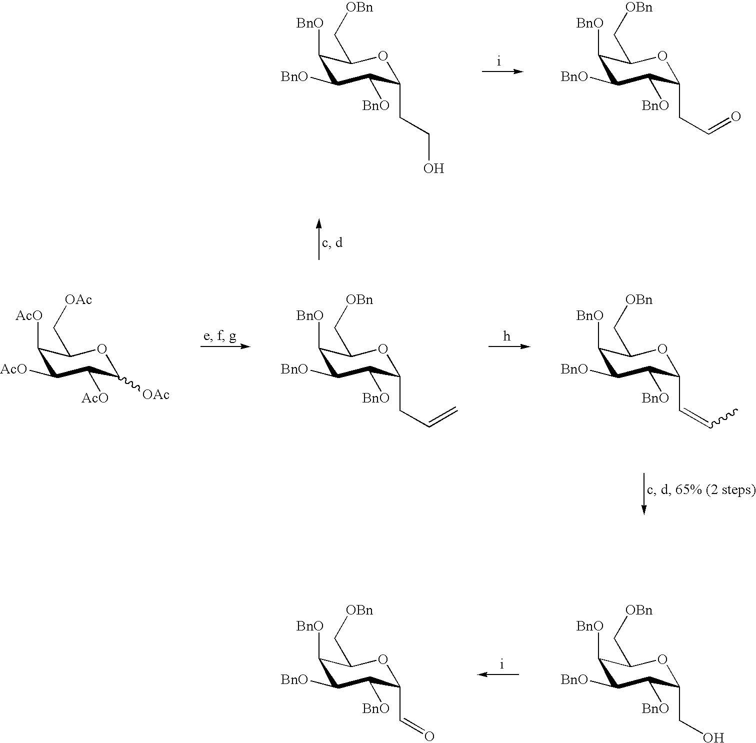
Conditions:

Step (c):
O₃, DCM, −78° C.;

Step (d):
NaBH₄, DCM, MeOH, the yield was 40% (3 steps);

Step (e):
allyltrimethylsilane (3.0 eq.), BF₃.OEt₂ (5.0 eq.), 0-10° C., 3 d, the yield was 77%;

Step (f):
NaOMe (0.1 eq.), MeOH, rt, 1 h;

Step (g):
NaH (2.0 eq.), BnBr (1.5 eq.), TBAI (cat.), DMF, THF, rt, 14 h, the yield was 93% (2 steps);

Step (h):
PdCl₂(PhCN)₂, benzene, reflux, 20 h, the yield was 73%; (i) (COCl)₂ (2.25 eq.), DMSO (5.50 eq.), DCM, −78° C., 0.5-1 h, then Et₃N (6.0 eq.), to 0° C., 2 h, the yield was 83%.

### Example C

### Julia-Kocienski Reaction

The reaction was carried as follows:

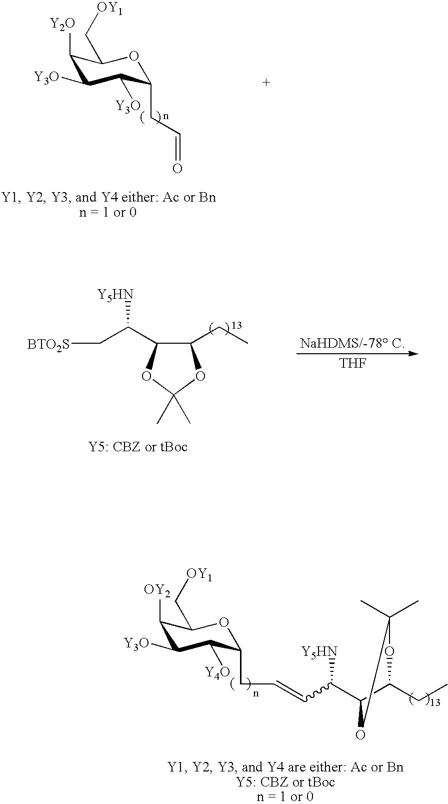

Alternatively, KHMDS or LiHDMS can be used instead of NaHDMS.

### Example D

### Synthesis of CRONY 101 with Product of Julia-Kocienski Reaction

The reaction was carried as follows: 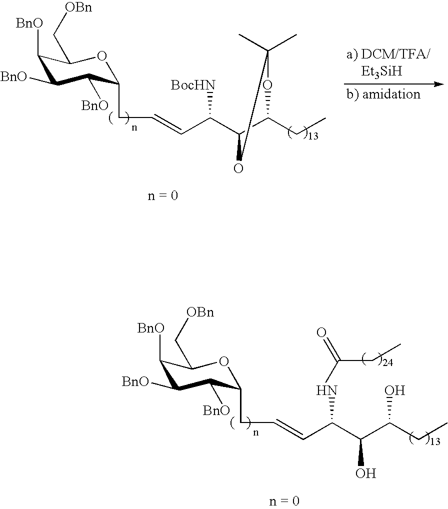
Conditions:

Step (a):
DCM/TFA(trifluoro acetic acid)/Et₃SiH (12:2:1), 0° C., 2 h;

Step (b):

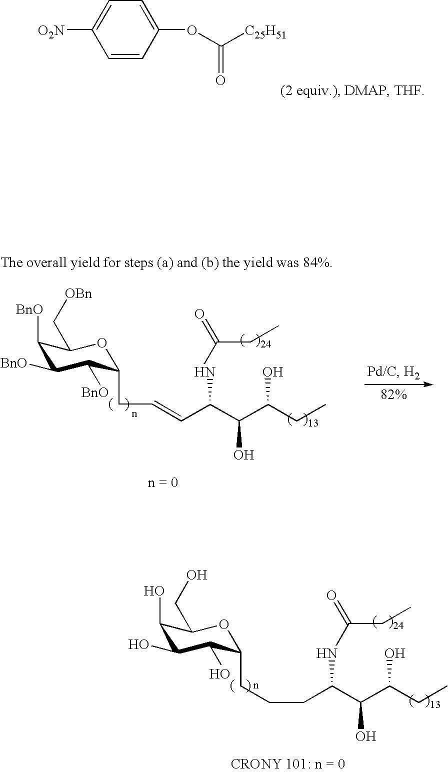

¹H NMR (500 MHz, pyridine-d5): δ8.43 (d, 1H, J=9.0 Hz), 6.65 (d, 1H, J=4.7 Hz), 6.49 (d, 1H, J=4.7 Hz), 6.37 (m, 2H), 6.16 (d, 1H, J=4.4 Hz), 5.98 (d, 1H, J=4.7 Hz), 5.12 (m, 1H), 4.72 (m, 1H), 4.52 (m, 3H), 4.36 (m, 1H), 4.22 (m, 4H), 2.72 (m, 1H), 2.58 (m, 1H), 2.45 (m, 2H), 2.32 (m, 2H), 2.22 (m, 1H), 1.93 (m, 2H), 1.85 (m, 2H), 1.70 (m, 1H), 1.48-1.17 (m, 68H), 0.89 (t, 6H, J=6.8 Hz).

### Example E

### Synthesis of GCM11i with Product of Julia-Kocienski Reaction

The reaction scheme was carried as follows: 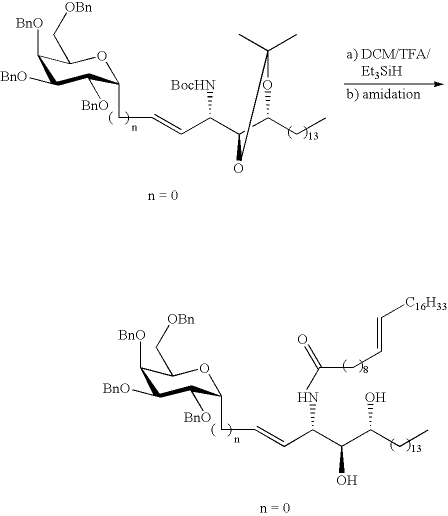
Conditions:

Step (a):
DCM/TFA/Et₃SiH (12:2:1), 0° C., 2 h;

Step (b): 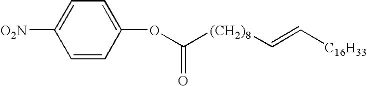
(2 equiv.), DMAP, THF.

The overall yield for steps (a) and (b) was 84%.

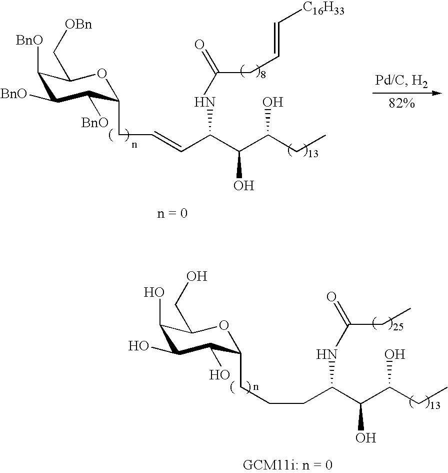

¹H NMR (500 MHz, pyridine-d5): δ8.43 (d, 1H, J=9.0 Hz), 6.65 (d, 1H, J=4.7 Hz), 6.49 (d, 1H, J=4.7 Hz), 6.37 (m, 2H), 6.16 (d, 1H, J=4.4 Hz), 5.98 (d, 1H, J=4.7 Hz), 5.12 (m, 1H), 4.72 (m, 1H), 4.52 (m, 3H), 4.36 (m, 1H), 4.22 (m, 4H), 2.72 (m, 1H), 2.58 (m, 1H), 2.45 (m, 2H), 2.32 (m, 2H), 2.22 (m, 1H), 1.93 (m, 2H), 1.85 (m, 2H), 1.70 (m, 1H), 1.48-1.17 (m, 68H), 0.87 (t, 6H, J=6.9 Hz).

### Example F

### Synthesis of GCK75a with Product of Julia-Kocienski Reaction

The reaction scheme was carried as follows: 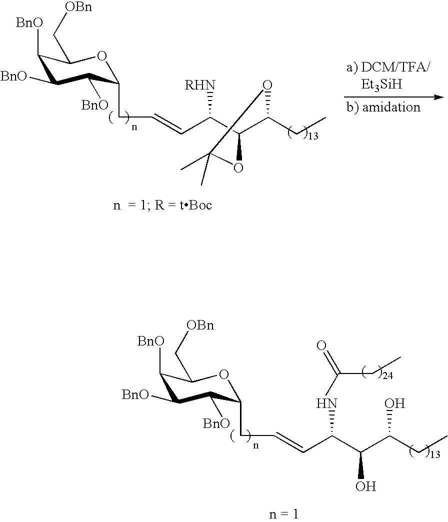
Conditions:

Step (a):
DCM/TFA/Et₃SiH (12:2:1), 0° C., 2 h;

Step (b):

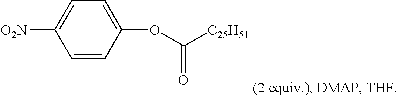

The overall yield for steps (a) and (b) was 83%.

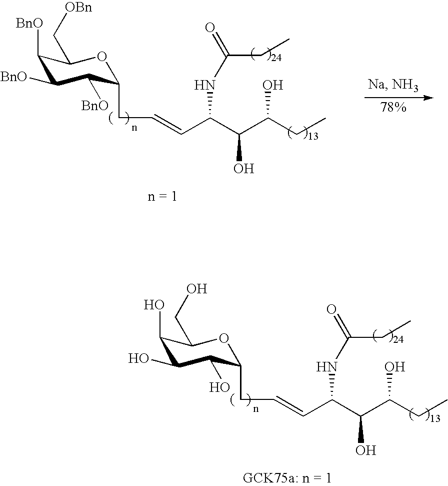

¹H NMR (500 MHz, pyridine-d5): δ8.39 (d, 1H, J=9.0 Hz), 6.76 (br s, 1H), 6.55 (br s, 1H), 6.44 (br s, 1H), 6.33 (m, 1H), 6.27 (m, 2H), 6.22 (br s, 1H), 6.11 (br s, 1H), 5.77 (m, 1H), 4.71 (m, 1H), 4.58 (m, 3H), 4.37 (m, 1H), 4.32 (m, 1H), 4.28 (m, 1H), 4.21 (m, 2H), 2.94 (m, 2H), 2.45 (t, 2H), 2.31 (m, 1H), 1.62 (m, 2H), 1.83 (m, 2H), 1.73 (m, 1H), 1.45-1.06 (m, 66H), 0.88 (t, 6H, J=6.9 Hz).

### Example G

### Synthesis of GCK75b with Product of Julia-Kocienski Reaction

The reaction scheme was carried as follows: 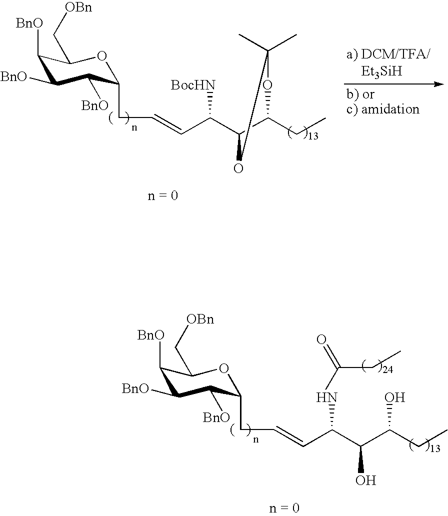
Conditions:

Step (a):
DCM/TFA/Et₃SiH (12:2:1), 0° C., 2 h;

Step (b): 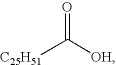
DIC (2 equiv.), HOBt (2 equiv.), DMAP, DMF, rt, 6 h, then Et₃N (2 equiv.), rt, overnight.

The overall yield for steps (a) and (b) was 80%.

Step (c): 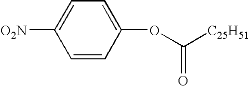
(2 equiv.), DMAP, THF.

The overall yield for steps (a) and (c) was 84%.

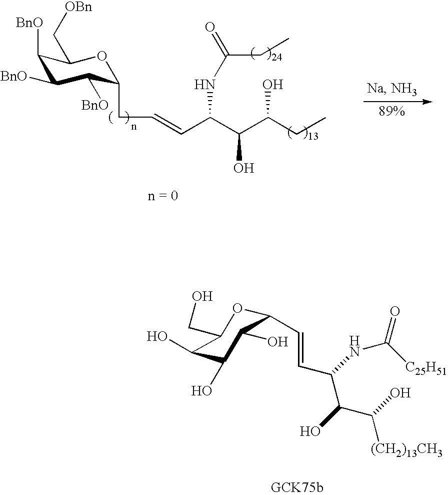

¹H NMR (500 MHz, CDCl₃/methanol-d4 (5:1)): δ5.73 (s, 2H), 4.56 (s, 1H), 4.38 (br s, 1H), 3.78 (m, 1H), 3.76 (s, 1H), 3.64 (s, 1H), 3.61 (ms, 1H), 3.55 (m, 1H), 3.41 (m, 1H), 3.24 (m, 2H), 2.04 (t, 1H, J=7.5 Hz), 1.56 (m, 1H), 1.45 (m, 2H), 1.37 (m, 1H), 1.24-1.00 (m, 68H), 0.72 (t, 6H, J=6.9 Hz).

### Example H

### Synthesis of Carbohydrate Counterpart for Olefin Metathesis

The reaction scheme was carried as follows: 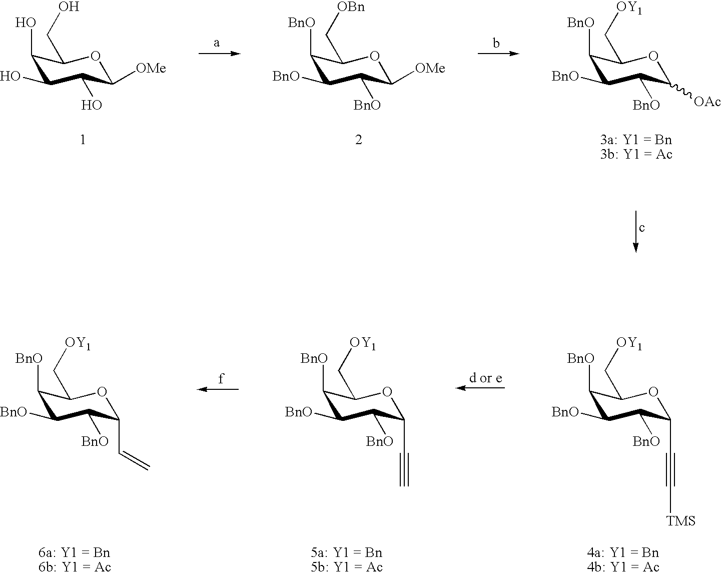
Conditions:

Step (a):
NaH (1.5 eq.), BnBr (1.25 eq.), TBAI (cat.), DMF, THF, rt, 24 h, the yield was 85%;

Step (b):
for 3a: Ac₂O (5.0 ml), HOAc (2.0 ml), aqueous H₂SO₄ (10%, 7 drops), rt, 1 d, the yield was 87%;
for 3b: Ac₂O (5.0 ml), HOAc (2.0 ml), aqueous H₂SO₄ (10%, 14 drops), rt, 10 mins, the yield was 99%;

Step (c):
for 4a: tributylstannyl(trimethylsilyl)acetylene (2.0 eq.), molecular sieve, DCM, 15 mins later, TMSOTf (2.0 eq.), rt, 1.5 h, the yield was 43%;
for 4b: the yield was 64%;

Step (d):
1 M NaOH (0.3 ml), MeOH, DCM, rt, 1 h, the yield was 99%;

Step (e):
KF.2H₂O (2.0 eq.), 18-crown-6 (1.0 eq), 70° C., 3 h;

Step (f):
for 6a: Lindlar reagent, H₂, ethyl acetate. The overall yield for steps (e) and (f) the yield was 94%;
for 6b: The overall yield for steps (e) and (f) was 92%.

### Example L

### Additional Synthesis of Carbohydrate Counterpart for Olefin Metathesis

The reaction scheme was carried as follows: 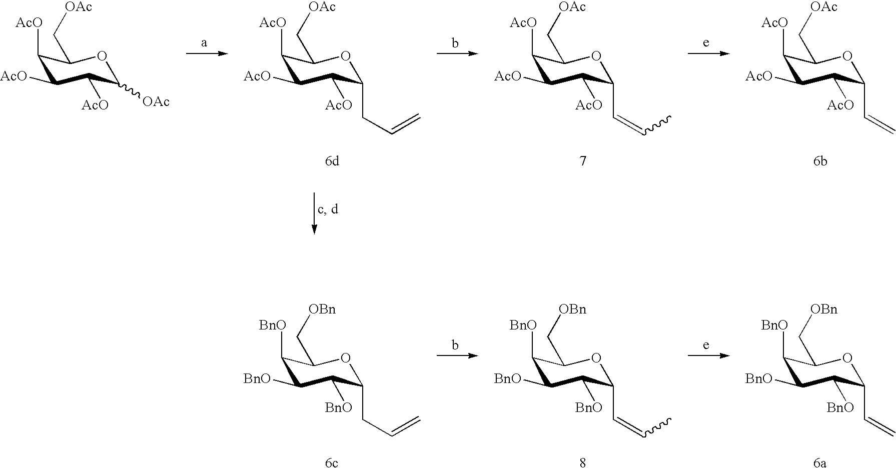
Conditions:

Step (a):
allyltrimethylsilane (3.0 eq.), BF₃.OEt₂ (5.0 eq.), 0-10° C., 3 d, the yield was 77%;

Step (b):
for perbenzyl protected: PdCl₂(PhCN)₂, benzene, reflux, 20 h, the yield was 73%;
for peracetyl protected: the yield was 87%;

Step (c):
NaOMe (0.1 eq.), MeOH, rt, 1 h;

Step (d):
NaH (2.0 eq.), BnBr (1.5 eq.), TBAI (cat.), DMF, THF, rt, 14 h.

The overall yield for steps (c) and (d) was 93%.

Step (e):
ethylene, 2^{nd} generation Grubbs catalyst (20 mol %), 3 d, the yield was 80-90%.

### Example J

### Synthesis of Lipid Side Chain Counterpart for Olefin Metathesis

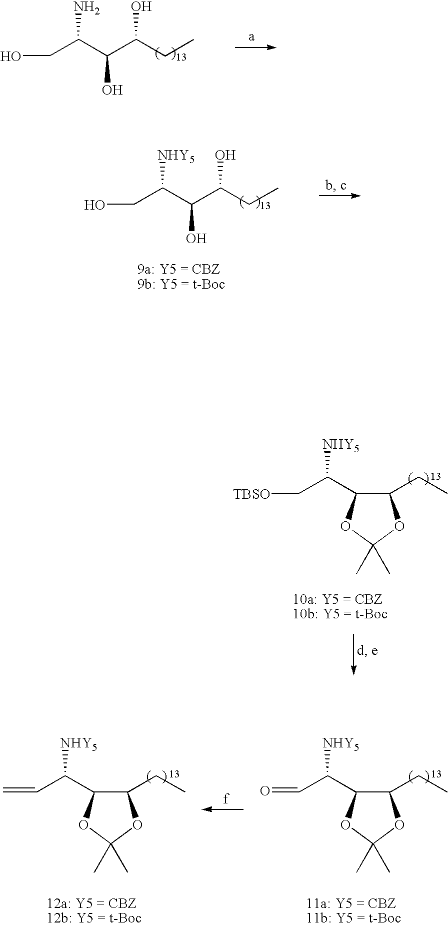 Conditions:

Step (a):
for benzyl carbamate: CBZCl (1.1 eq.), 1N NaHCO₃, 1,4-dioxane, ethyl acetate, rt, overnight, the yield was 90%;
for t-butyl carbamate: 1N NaOH (1.5 eq.), (t-Boc)₂O (1.5 eq.), ethanol, water, rt, 1 h;

Step (b):
for benzyl carbamate: TBSCl (1.2 eq.), Et₃N (1.1 eq.), 4-DMAP (0.05 eq.), DCM, DMF, 0° C., 1 h, the yield was 96%;
for t-butyl carbamate: The overall yield for steps (a) and (b) was 93%;

Step (c):
for benzyl carbamate: 2,2-dimethoxypropane (5-10 eq.), PPTs (0.07 eq.), DCM, rt, 2 h, the yield was 99%;
for t-butyl carbamate: directly used for next step without purification;

Step (d):
for benzyl carbamate: TBAF (2.0 eq.), HOAc (trace), THF, rt, 4 h, the yield was 99%;
for t-butyl carbamate: TBAF (1.2 eq.), THF, 0° C., rt, 3 h, the overall yield for steps (c) and (d) was 94%;

Step (e):
for benzyl carbamate: (COCl)₂ (3.0 eq.), DMSO (9.0 eq.), DCM, −78° C., 0.5 h, then Et₃N (6.0 eq.), rt, 10 mins, the yield was 44%; or polymer-supported oxidant (2.7 eq), TEMPO (cat.), DCM, 0° C., 2.5 h;
for t-butyl carbamate: (COCl)₂ (2.5 eq.), DMSO (6.0 eq.), DCM, −60° C., 45 mins, then Et₃N (7.0 eq.), −60° C. to −30° C., 2 h;

Step (f):
for benzyl carbamate: Tebbe reagent (0.5 M in tol., 1.2 eq.), −70° C.-−50° C., 4 h, 54% (2 steps);
for t-butyl carbamate: Tebbe reagent (0.5 M in tol., 1.28 eq.), −70° C., 2 h, then −15° C., 1 h, the yield was 68% (2 steps).

### Example K

### Olefin Metathesis

The reaction scheme was carried as follows: 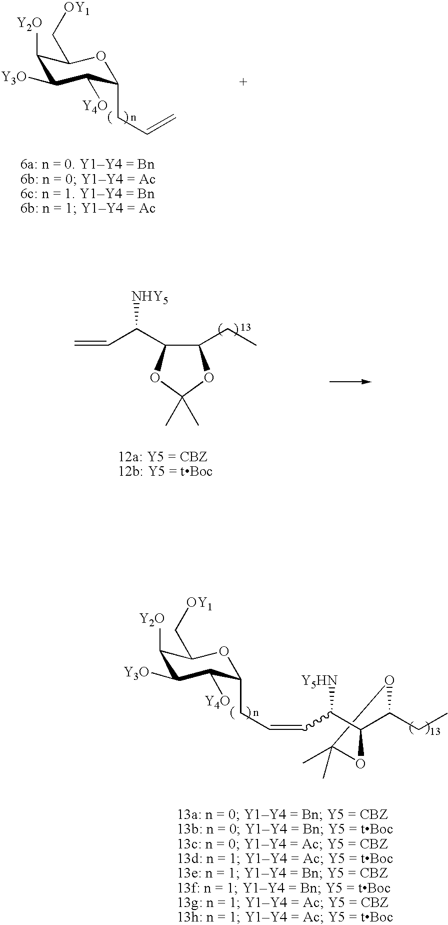
Conditions (for Representative Examples):
for 13a:
   6a (3 equiv.), Grubbs catalyst (2^{nd}, 15 mol %), benzene, 50-60° C., 1d, 37% (only E isomer);
for 13b:
   6b (1.5 equiv.), Hoveyda-Grubbs catalyst (2^{nd}, 15 mol %), benzene, 60° C., 1 d, 23% (only E isomer);
for 13f:
   6c (1.5 equiv.), 2^{nd} Grubbs catalyst (15 mol %), DCM, reflux, 1 d, 61% (E/Z 5:1).

### Example L

### Synthesis of CRONY 101 with Product of Olefin Metathesis

The reaction scheme was carried as follows: 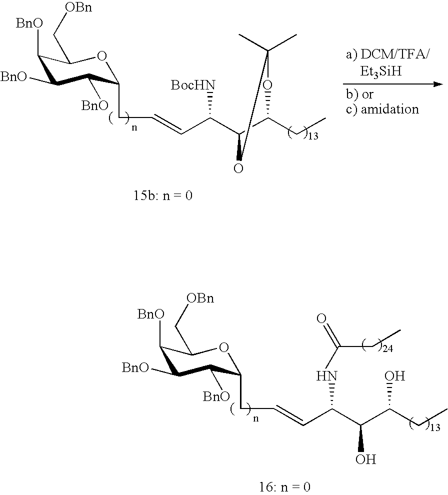
Conditions:

Step (a):
DCM/TFA/Et₃SiH (12:2:1), 0° C., 2 h;

Step (b): 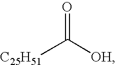
DIC (2 equiv.), HOBt (2 equiv.), DMAP, DMF, RT, 6 h, then Et₃N (2 equiv.), RT, overnight (the overall yield for steps (a) and (b) was 80%);

Step (c): 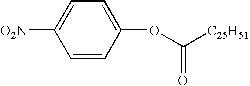
(2 equiv.), DMAP, THF (The overall yield for steps (a) and (c) was 84%).

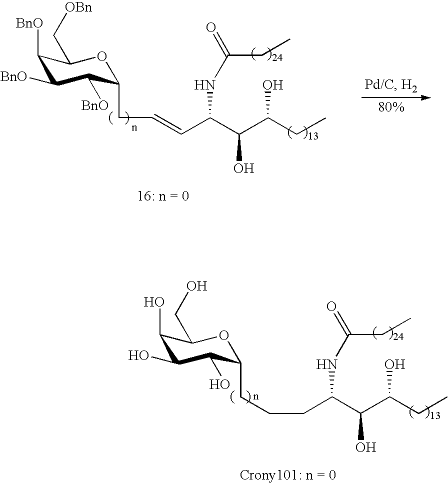

¹H NMR (500 MHz, pyridine-d5): δ8.43 (d, 1H, J=9.0 Hz), 6.65 (d, 1H, J=4.7 Hz), 6.49 (d, 1H, J=4.7 Hz), 6.37 (m, 2H), 6.16 (d, 1H, J=4.4 Hz), 5.98 (d, 1H, J=4.7 Hz), 5.12 (m, 1H), 4.72 (m, 1H), 4.52 (m, 3H), 4.36 (m, 1H), 4.22 (m, 4H), 2.72 (m, 1H), 2.58 (m, 1H), 2.45 (m, 2H), 2.32 (m, 2H), 2.22 (m, 1H), 1.93 (m, 2H), 1.85 (m, 2H), 1.70 (m, 1H), 1.48-1.17 (m, 68H), 0.89 (t, 6H, J=6.8 Hz).

### Example M

### Synthesis of GCM11i with Product of Olefin Metathesis

The reaction scheme was carried as follows: 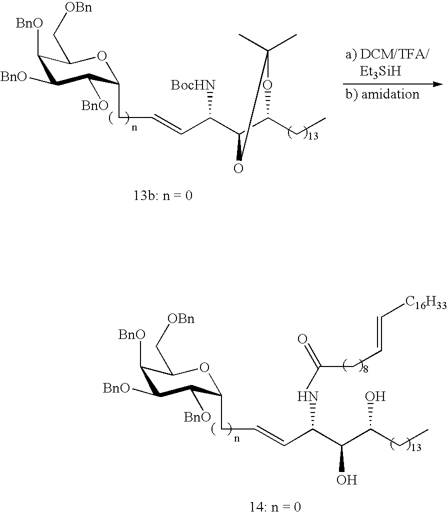
Conditions:

Step (a):
DCM/TFA/Et₃SiH (12:2:1), 0° C., 2 h;

Step (b): 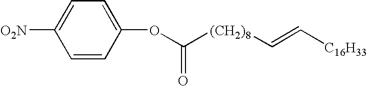
(2 equiv.), DMAP, THF.

The overall yield for steps (a) and (b) was 84%.

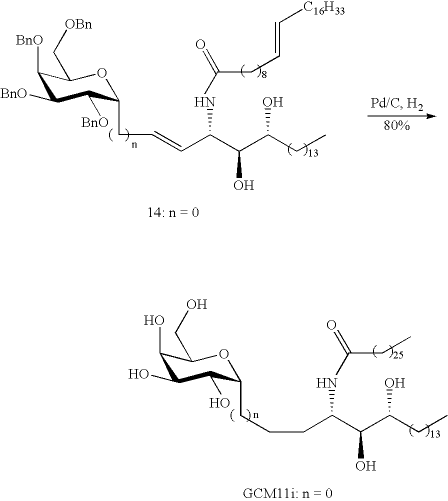

¹H NMR (500 MHz, pyridine-d5): δ8.43 (d, 1H, J=9.0 Hz), 6.65 (d, 1H, J=4.7 Hz), 6.49 (d, 1H, J=4.7 Hz), 6.37 (m, 2H), 6.16 (d, 1H, J=4.4 Hz), 5.98 (d, 1H, J=4.7 Hz), 5.12 (m, 1H), 4.72 (m, 1H), 4.52 (m, 3H), 4.36 (m, 1H), 4.22 (m, 4H), 2.72 (m, 1H), 2.58 (m, 1H), 2.45 (m, 2H), 2.32 (m, 2H), 2.22 (m, 1H), 1.93 (m, 2H), 1.85 (m, 2H), 1.70 (m, 1H), 1.48-1.17 (m, 68H), 0.87 (t, 6H, J=6.9 Hz).

### Example N

### Synthesis of GCK75a with Product of Olefin Metathesis

The reaction scheme was carried as follows: 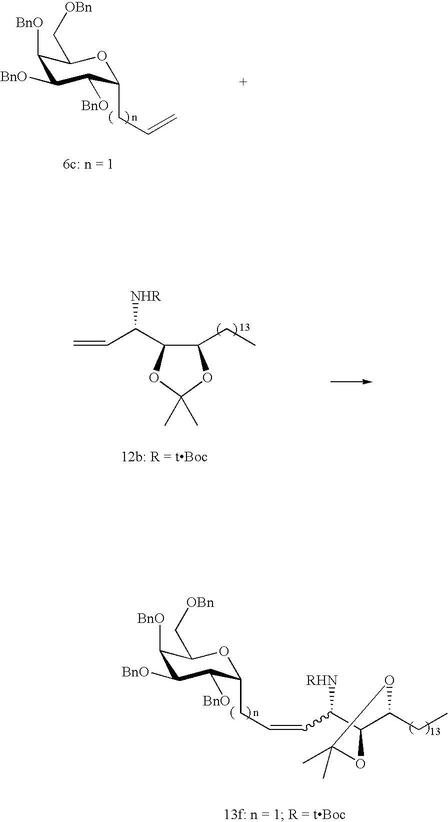
Conditions:

6c (1.5 equiv.), Grubbs catalyst (2^{nd}, 15 mol %), DCM, reflux, 1d, the yield was 61% (E/Z 5:1). 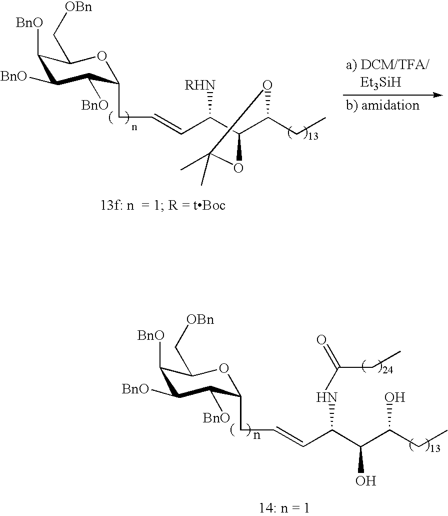
Conditions:

Step (a):
DCM/TFA/Et₃SiH (12:2:1), 0° C., 2 h;

Step (b): 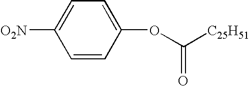
(2 equiv.), DMAP, THF.

The overall yield for steps (a) and (b) was 83%.

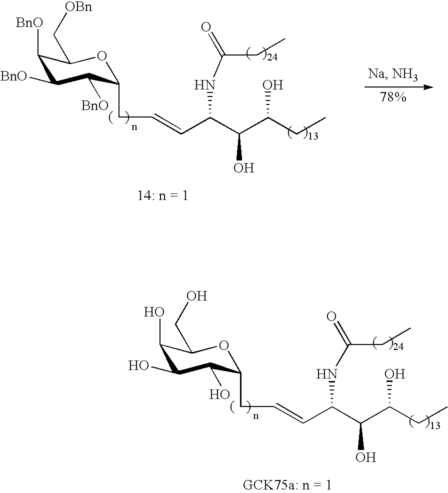

¹H NMR (500 MHz, pyridine-d5): δ8.39 (d, 1H, J=9.0 Hz), 6.76 (br s, 1H), 6.55 (br s, 1H), 6.44 (br s, 1H), 6.33 (m, 1H), 6.27 (m, 2H), 6.22 (br s, 1H), 6.11 (br s, 1H), 5.77 (m, 1H), 4.71 (m, 1H), 4.58 (m, 3H), 4.37 (m, 1H), 4.32 (m, 1H), 4.28 (m, 1H), 4.21 (m, 2H), 2.94 (m, 2H), 2.45 (t, 2H), 2.31 (m, 1H), 1.62 (m, 2H), 1.83 (m, 2H), 1.73 (m, 1H), 1.45-1.06 (m, 66H), 0.88 (t, 6H, J=6.9 Hz).

### Example O

### Synthesis of GCK75b with Product of Olefin Metathesis

The reaction scheme was carried as follows: 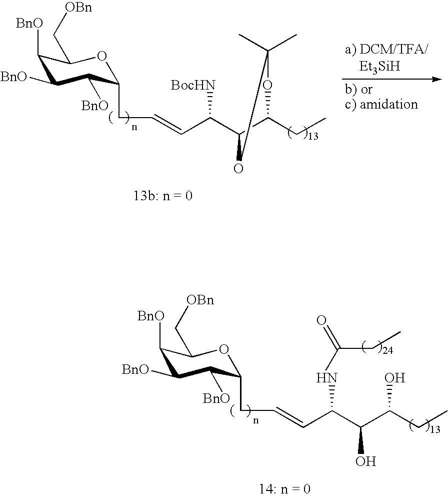
Conditions:

Step (a):
DCM/TFA/Et₃SiH (12:2:1), 0° C., 2 h;

Step (b): 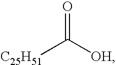
DIC (2 equiv.), HOBt (2 equiv.), DMAP, DMF, RT, 6 h, then Et₃N (2 equiv.), RT, overnight (The overall yield for steps (a) and (b) was 80%);

Step (c): 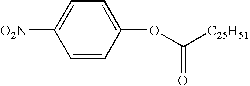
(2 equiv.), DMAP, THF (The overall yield for steps (a) and (c) was 84%).

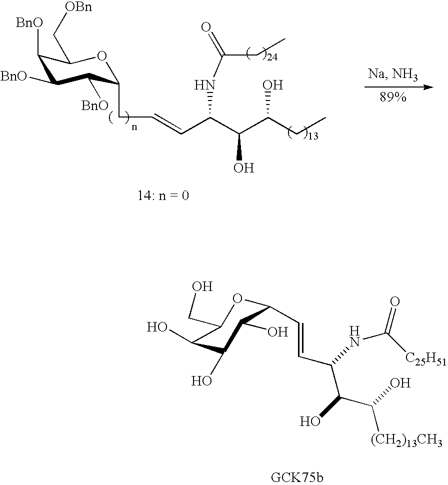

¹H NMR (500 MHz, CDCl₃/methanol-d4 (5:1)): δ5.73 (s, 2H), 4.56 (s, 1H), 4.38 (br s, 1H), 3.78 (m, 1H), 3.76 (s, 1H), 3.64 (s, 1H), 3.61 (ms, 1H), 3.55 (m, 1H), 3.41 (m, 1H), 3.24 (m, 2H), 2.04 (t, 1H, J=7.5 Hz), 1.56 (m, 1H), 1.45 (m, 2H), 1.37 (m, 1H), 1.24-1.00 (m, 68H), 0.72 (t, 6H, J=6.9 Hz).

### Example P

### Synthesis of

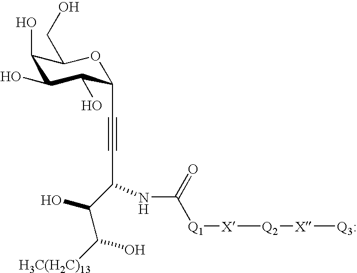

The reaction scheme is carried out as follows:

### Example Q

### Synthesis of

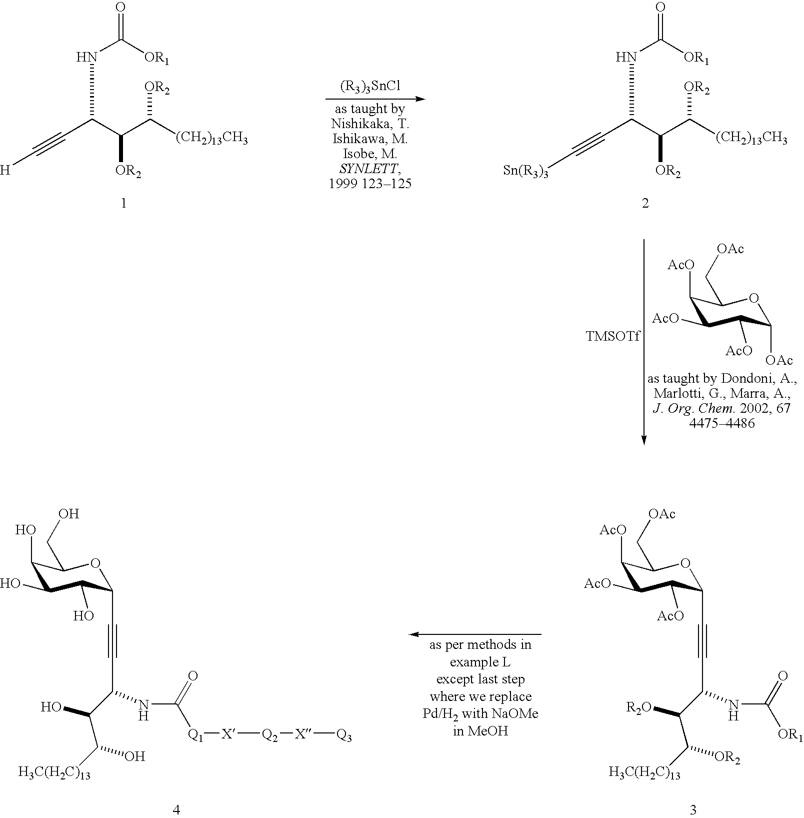

The reaction scheme is carried out as follows: 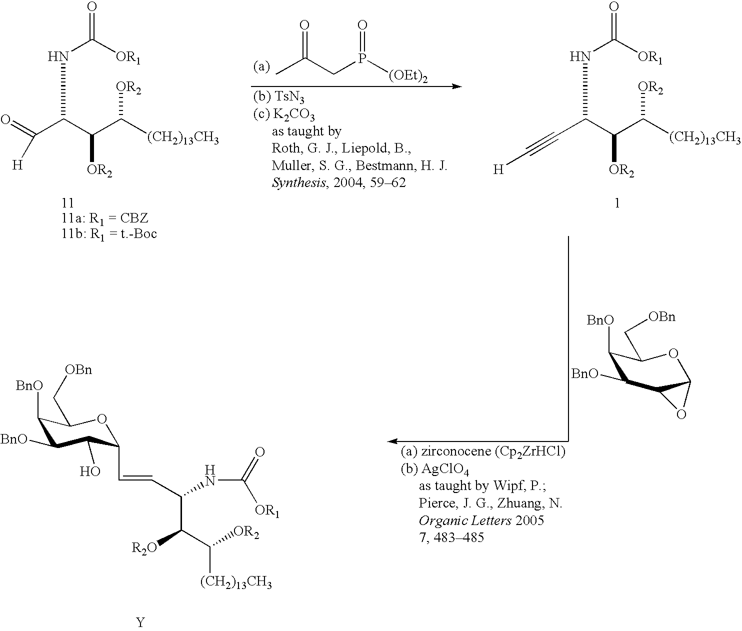
Biological Assays and Data

The following Biological Example illustrates the invention without limiting its scope.

### Example 1

### Immunological Characterization of Compounds GCM11i and GCK75(b) Materials and Methods

α-Galactosylceramide (α-GalCer, KRN or KRN7000) was synthesized by Kirin Brewery (Gumma, Japan). The stock solution was dissolved in a 0.5% polysorbate −20 (Nikko Chemical, Tokyo), 0.9% NaCl solution at a concentration of 200 μg/ml, and diluted in PBS just before injection into mice. α-C-galactosylceramide (α-C-GalCer, CRONY or CRONY-101) was synthesized as described in commonly owned U.S. patent application Ser. No. 10/462,211. Compounds GCM1 μl, GCK75(a) and GCK75(b) were synthesized as described herein. The stock solution was originally dissolved in 100% DMSO at a concentration of 1 mg/ml. Before injection into mice, it was diluted to a concentration of 200 μg/ml in a 0.5% polysorbate-20 (Nikko Chemical, Tokyo), 0.9% NaCl solution, and diluted in PBS just before injection into mice.

Six to eight-week-old female BALB/c mice were purchased from the National Cancer Institute (Bethseda, Md.). All mice were maintained under pathogen-free conditions.

The serum concentrations of IFN-γ were measured at 2, 6, 12, and 24 hours after treatment with α-GalCer, α-C-GalCer, compound GCM1 μl, GCK75(a), GCK75(b), or nothing using a sandwich ELISA (e-bioscience, San Diego).

Plasmodium yoelii (17NXL strain) was maintained by alternate cyclic passages in Anopheles stephensi mosquitoes and Swiss Webster mice. Sporozoites obtained from dissected salivary glands of infected mosquitoes were used for challenge of the mice. Challenge of mice to determine the development of liver-stage malaria infection was performed by an intravenous injection of 10,000 viable sporozoites into the tail vein, which was performed two days after the mice were injected intravenously (i.v.) with 1 μg of each of α-C-GalCer (CRONY), compound GCM1 μl, GCK75(b), or nothing. The outcome of the challenge was determined 42 hours later by measuring the parasite burden (i.e., by quantifying the amount of P. yoelii-specific 18S rRNA molecules) in the livers of the mice using a quantitative real-time RT-PCR method, as taught in Bruna-Romero et al., Int. J. Parasitol. 31, 1449-1502, 2001. Specifically, a 2 μg sample of total RNA prepared from the livers of challenged mice was reverse-transcribed, and an aliquot of the resulting cDNA (133 ng) was used for quantitative real-time PCR amplification of P. yoelii 18S rRNA sequences. This amplification was performed in a GeneAmp® 5700 Sequence Detection System (PE Applied Biosystems, Foster City, Calif.). For this purpose, primers 5′-GGGGATTGGTTTTGACGTTTTTGCG-3′ (54 nM) and 5′AAGCATTAAATAAAGCGAATACATCCTTAT-3′ (60 nm) were used, together with the dsDNA-specific dye SYBR Green I incorporated into the PCR reaction buffer (PE Biosystems, Foster City, Calif.) in order to detect the PCR product generated. The temperature profile of the reaction was 95° C. for 10 minutes followed by 35 cycles of denaturation of 95° C. for 15 seconds and annealing/extension at 60° C. for 1 minute.

### Results and Discussion

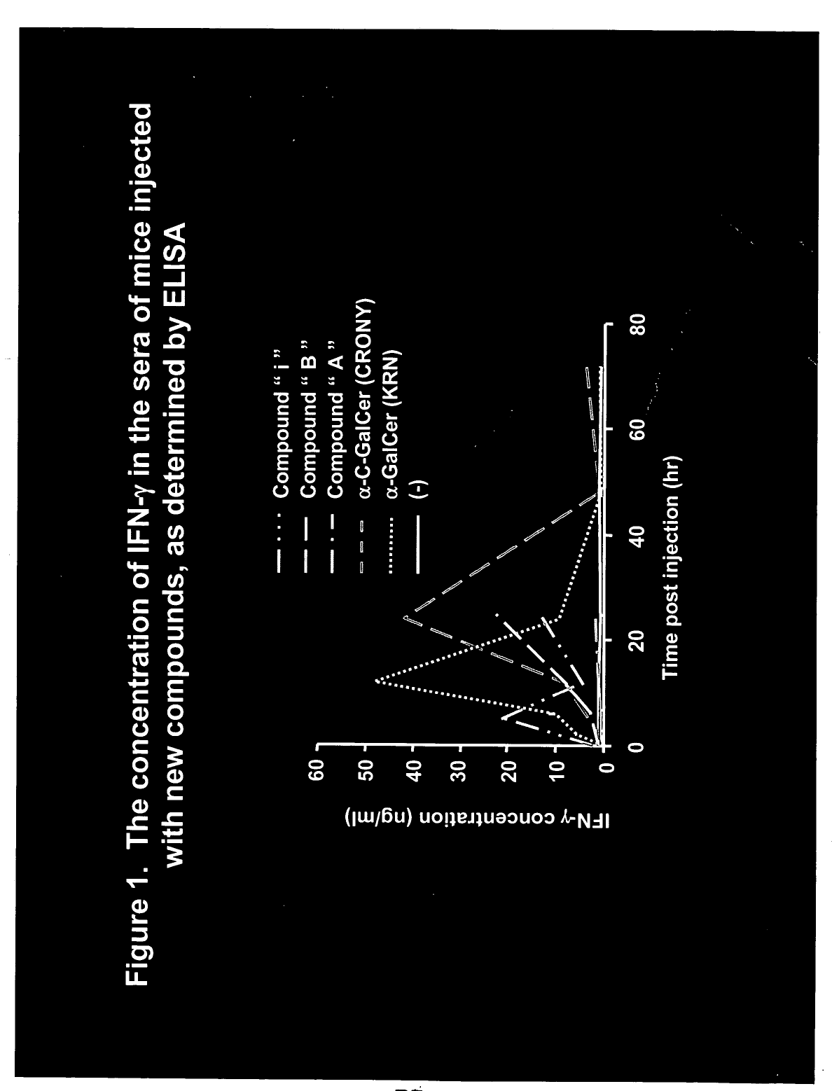

To compare the timing of immunological responses to α-GalCer (KRN), α-C-GalCer (CRONY), GCM11i, GCK75(a), and GCK75(b), mice were injected intravenously (i.v.) with 1 μg of each of the glycolipids or with nothing. At 2, 6, 12, and 24 hours post-injection, IFN-γ concentrations in the sera were measured by ELISA. As shown in FIG. 1, α-GalCer (KRN) or α-C-GalCer (CRONY) administration induced IFN-γ production in the sera, peaking at 12 or 24 hours post-injection, respectively. Surprisingly, GCM1 μl induced a peak IFN-γ response at 6 hours post-injection, whereas GCK75(b) induced the peak response more than 24 hours post-injection. The level of the peak response of GCM1 μl and GCK75(b) were lower than that of α-GalCer and α-C-GalCer.

As GCM11i induced a peak IFN-γ response much earlier than the rest of the α-GalCer analogs tested, it is likely that GCM11i may activate NKT cells and induce maturation of dendritic cells (DCs) more acutely. Since antigen presentation by antigen-presenting cells, such as DCs, to specific T cells and the generation of protective immune response normally occur fairly quickly, i.e., in 4-12 hours, an adjuvant based on compound GCM11l may have superior properties. Also, for therapeutic purposes, it would be even more efficient to use an adjuvant that activates NKT cells quickly (e.g., GCM11i) and another that activates NKT cells much later (e.g., GCK75(b)), because the combined use of such adjuvants would have both acute and prolonged biological activity against pathogens and various diseases, including cancer, allergy and various infectious diseases such as hepatitis B and C.

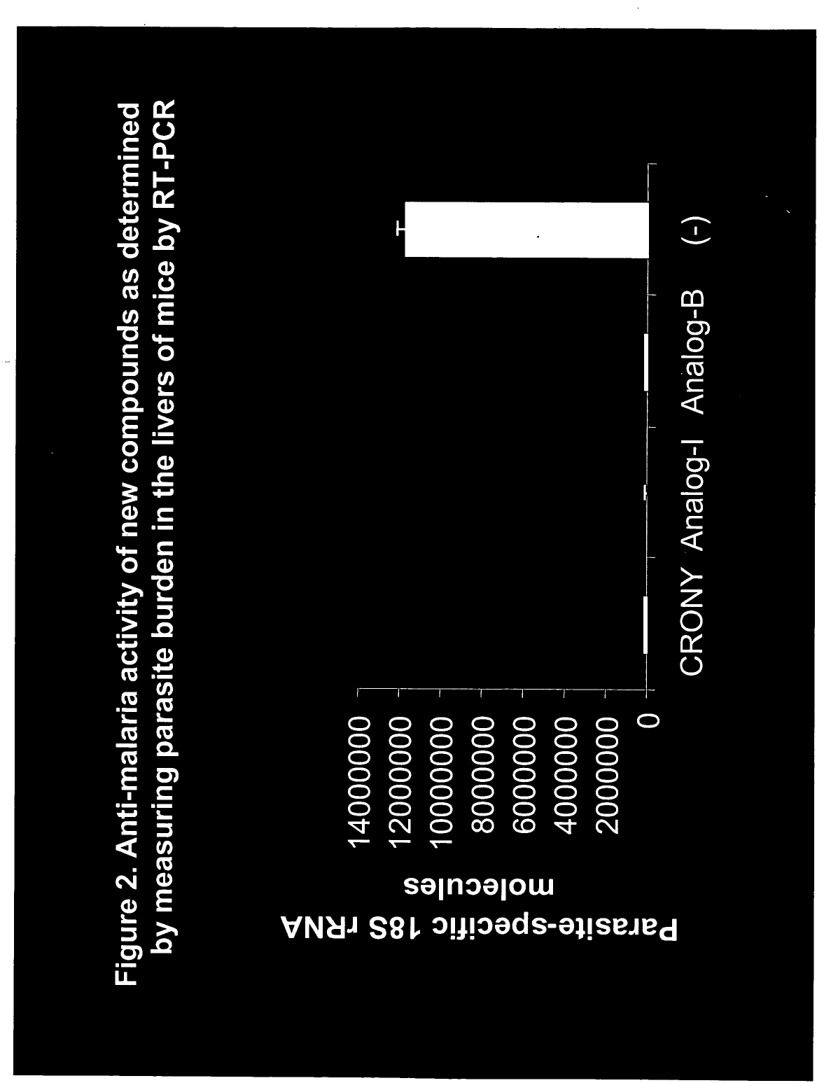

Since the present inventors found a significant level of IFN-γ being produced by compounds GCM11i and GCK75(b), these two glycolipids were further tested to determine their anti-malarial activity in vivo. For this purpose, mice were injected intravenously with 1 μg of, either, GCM11i or GCK75(b), α-C-GalCer (CRONY) (positive control, see commonly owned U.S. patent application Ser. No. 10/462,211), or nothing (negative control), and two days later the injected mice were challenged with 10,000 Plasmodium yoelii sporozoites. Forty two hours after the parasite challenge, livers were collected and the amounts of parasite-specific 18S rRNA were determined in the livers by a quantitative real-time RT-PCR assay. As shown in FIG. 2, both GCM1 μl and GCK75(b) displayed a level of anti-malarial activity comparable to that of α-C-GalCer (CRONY), almost completely inhibiting the development of parasites in the livers.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all values are approximate, and are provided for description.

All patents, applications, publications, test methods, literature, and protocols cited throughout this application, are incorporated herein by reference entireties for all purposes. In case of a conflict between material incorporated by reference and the present specification, the present specification controls.

1. A compound of formula (I) 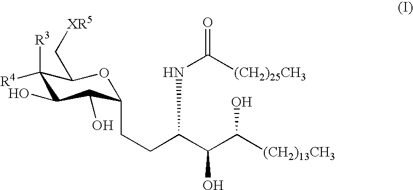
wherein X is O or NH;
R³ is OH or a monosaccharide and R⁴ is hydrogen, or R³ is hydrogen and R⁴ is OH or a monosaccharide;
R⁵ is hydrogen or a monosaccharide;
and pharmaceutically acceptable salts or esters thereof.

2. The compound of claim 1 described by formula (I-a)

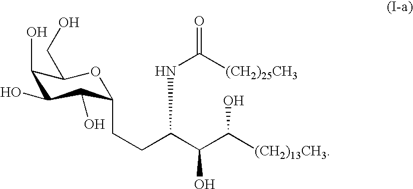

3. A compound of formula (II) 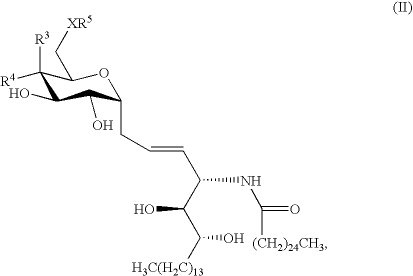
wherein X is O or NH;
R³ is OH or a monosaccharide and R⁴ is hydrogen, or R³ is hydrogen and R⁴ is OH or a monosaccharide;
R⁵ is hydrogen or a monosaccharide;
and pharmaceutically acceptable salts or esters thereof.

4. The compound of claim 3 described by formula (II-a)

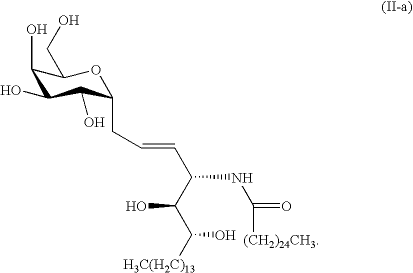

5. A compound of formula (III) 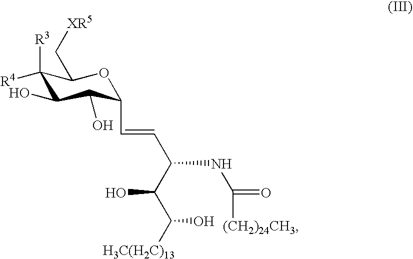
wherein X is O or NH;
R³ is OH or a monosaccharide and R⁴ is hydrogen, or R³ is hydrogen and R⁴ is OH or a monosaccharide;
R⁵ is hydrogen or a monosaccharide;
and pharmaceutically acceptable salts or esters thereof.

6. The compound of claim 5 described by formula (III-a)(cis)

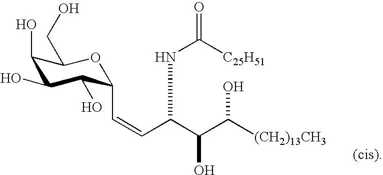

7. The compound of claim 5 described by formula (III-a)(trans):

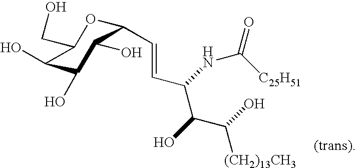

8. A method of treating a disease in a mammal in need thereof, comprising administering to said mammal a therapeutically effective amount of a compound of any one of claims 1-7.

9. The method of claim 8, wherein said disease is selected from the group consisting of infection, cancer and autoimmune disease.

10. The method of claim 8, wherein said disease is malaria.

11. The method of claim 8, wherein said mammal is a human.

12. A method of inducing the production of Th1 type cytokine in a mammal in need thereof, by administering to said mammal a therapeutically effective amount of a compound of any one of claims 1-7.

13. The method of claim 12, wherein said Th1 type cytokine is IFN-γ.

14. A method for augmenting the immunogenicity of an antigen in a mammal, comprising immunizing the mammal with an adjuvant comprising a therapeutically effective amount of a compound of any one of claims 1-7.

15. The method of claim 14, wherein said antigen is malaria-specific.

16. The method of claim 15, wherein said malaria-specific antigen comprises irradiated plasmodial sporozoites.

17. A method of making a compound of formula A: 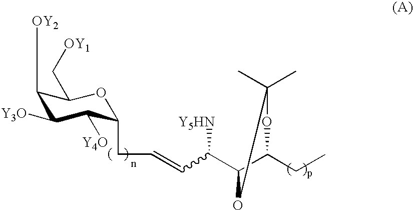
comprising the step of reacting 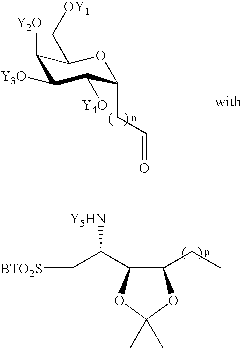
and a heterocyclic sulfone;
wherein
Y₁, Y₂, Y₃, and Y₄ are each independently protecting groups for sugar;
Y₅ is a protecting group for nitrogen;
n is 1 or 0; and
p is an integer from 1-100.

18. The method of claim 17, wherein
Y₁, Y₂, Y₃, and Y₄ are each independently selected from the group consisting of Ac (acetyl), Bn (benzyl), Bz (benzoate), PMB (para methoxybenzyl), TBDMS (tertiarybutyldimethylsilyl), TBDPS (tertiarybutyldiphenylsilyl), or connecting the oxygens of C4 and C6 with benzylidene or paramethoxybenzylidene.

19. The method of claim 18, wherein Y₁, Y₂, Y₃, and Y₄ are each independently Ac or Bn.

20. The method of claim 17, wherein Y₅ is selected from the group consisting of CBZ, t-Boc, FMOC (fluorenylmethyleneoxycarbonyl), and Phth (phthaloyl).

21. The method of claim 20, wherein Y₅ is CBZ or t-Boc.

22. The method of claim 17, wherein p is 13.

23. A method of making a compound of formula (B) 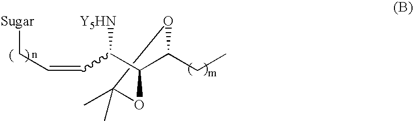
comprising the step of reacting 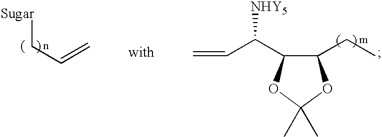
wherein
the sugar moiety can be protected or un-protected;
n is an integer from 0 to 20,
m is an integer from 1-100; and
Y₅ is a protecting group for nitrogen.

24. The method of claim 23 the sugar is protected and selected from the group consisting of galactose, glucose, glucosamine, mannose, galactosamine, fucose, and rhamnose.

25. The method of claim 24, wherein the sugar moiety is a protected galactose.

26. The method of claim 23, wherein the sugar moiety is 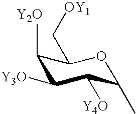
wherein Y₁, Y₂, Y₃, and Y₄ are each independently protecting groups for sugar.

27. The method of claim 23, wherein n is 1 or 0.

28. The method of claim 23, wherein m is 13.

29. The method of claim 26, wherein Y₁, Y₂, Y₃, and Y₄ are each independently selected from the group consisting of Ac (acetyl), Bn (benzyl), Bz (benzoate), PMB (para methoxybenzyl), TBDMS (tertiarybutyldimethylsilyl), TBDPS (tertiarybutyldiphenylsilyl), connecting the oxygens of C4 and C6 with benzylidene or paramethoxybenzylidene.

30. The method of claim 29, wherein Y₁, Y₂, Y₃, and Y₄ are each independently Ac or Bn.

31. The method of claim 26, wherein Y₅ is selected from the group consisting CBZ, t-Boc, FMOC (fluorenylmethyleneoxycarbonyl), and Phth (phthaloyl).

32. The method of claim 31, wherein Y₅ is CBZ or t-Boc.

33. A method of synthesizing glycolipid comprising the steps of:
(a) reacting 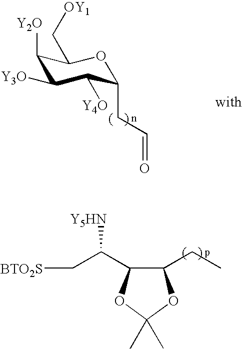
and a heterocyclic sulfone;
to form a compound of formula (A) 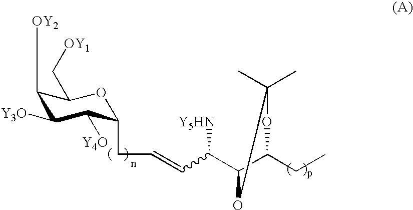
wherein
Y₁, Y₂, Y₃, and Y₄ are each independently protecting groups for sugar;
Y₅ is a protecting group for nitrogen;
n is 1 or 0; and
p is an integer from 1-100; and
(b) further reacting the compound of formula (A) to form a glycolipid of the formula (C): 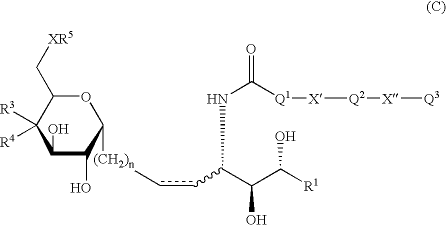
wherein X is O or NH;
n is 1 or 0;
R¹ is selected from the group consisting of —(CH₂)₁₁CH₃, —(CH₂)₁₂CH₃, —(CH₂)₁₃CH₃, —(CH₂)₉CH(CH₃)₂, —(CH₂)₁₀CH(CH₃)₂, —(CH₂)₁₁CH(CH₃)₂ and —(CH₂)₁₁CH(CH₃)—C₂H₅;
R³ is OH or a monosaccharide and R⁴ is hydrogen, or R³ is hydrogen and R⁴ is OH or a monosaccharide;
R⁵ is hydrogen or a monosaccharide;
Q¹ is optionally present and is a C₁₋₁₀ straight or branched chain alkylene, alkenylene, or alkynylene;
X′ is optionally present and is O, S or NR⁸;
Q² is optionally present and is a C₁₋₁₀ straight or branched chain alkylene, alkenylene or alkynylene;
X″ is optionally present and is O, S or NR⁸;
Q³ is a straight or branched chain C₁₋₁₀ alkyl, alkenyl or alkynyl, or is hydrogen,
wherein each Q¹, Q² or Q³ is optionally substituted with hydroxyl, halogen, cyano, nitro, SO₂, NHR⁸, or C(═O)—R⁹; and wherein
R⁸ is hydrogen, C₁₋₅ alkyl, C₁₋₅ alkoxy, halogen, cyano, nitro, SO₂ or C(═O)—R⁹;
R⁹ is hydrogen, C₁₋₅ alkyl, C₁₋₅ alkoxy or NHR¹⁰;
R¹⁰ is hydrogen, C₁₋₅ alkyl or C₁₋₅ alkoxy;
and pharmaceutically acceptable salts or esters thereof.

34. The method of claim 33 wherein the compound of formula (C) is

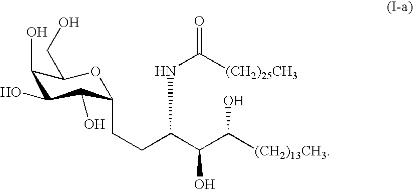

35. The method of claim 33 wherein the compound of formula (C) is

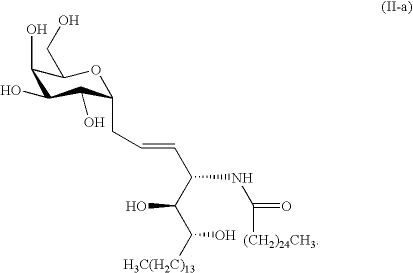

36. The method of claim 33 wherein the compound of formula (C) is

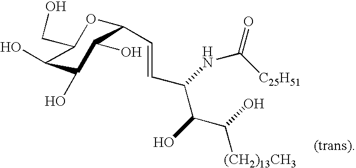

37. The method of claim 33 wherein the compound of formula (C) is

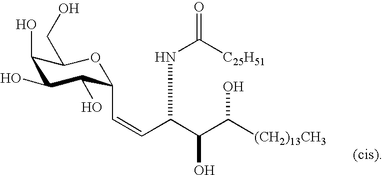

38. The method of claim 33 wherein the compound of formula (C) is

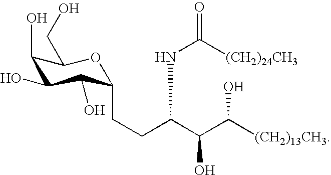

39. A method of synthesizing glycolipid comprising the steps of:
(a) reacting 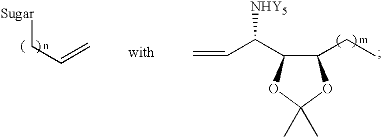
to form a compound of formula (B) 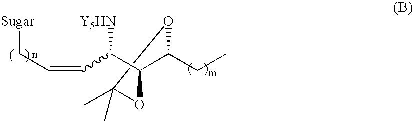
wherein
the sugar moiety can be protected or un-protected;
n is an integer from 0 to 20,
m is an integer from 1-100; and
Y₅ is a protecting group for nitrogen.
(b) further reacting the compound of formula (B) to form a glycolipid of the formula (C): 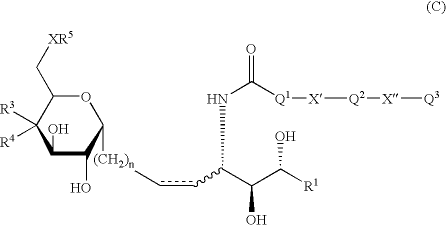
wherein X is O or NH;
n is 1 or 0;
R¹ is selected from the group consisting of —(CH₂)₁₁CH₃, —(CH₂)₁₂CH₃, —(CH₂)₁₃CH₃, —(CH₂)₉CH(CH 3)₂, —(CH₂)₁₀CH(CH₃)₂, —(CH₂)₁₁CH(CH₃)₂ and —(CH₂)₁₁CH(CH₃)—C₂H₅;
R³ is OH or a monosaccharide and R⁴ is hydrogen, or R³ is hydrogen and R⁴ is OH or a monosaccharide;
R⁵ is hydrogen or a monosaccharide;
Q¹ is optionally present and is a C₁₋₄₀ straight or branched chain alkylene, alkenylene, or alkynylene;
X′ is optionally present and is O, S or NR⁸;
Q² is optionally present and is a C₁₋₁₀ straight or branched chain alkylene, alkenylene or alkynylene;
X″ is optionally present and is O, S or NR⁸;
Q³ is a straight or branched chain C₁₋₁₀ alkyl, alkenyl or alkynyl, or is hydrogen,
wherein each Q¹, Q² or Q³ is optionally substituted with hydroxyl, halogen, cyano, nitro, SO₂, NHR⁸, or C(═O)—R⁹; and wherein
R⁸ is hydrogen, C₁₋₅ alkyl, C₁₋₅ alkoxy, halogen, cyano, nitro, SO₂ or C(═O)—R⁹;
R⁹ is hydrogen, C₁₋₅ alkyl, C₁₋₅ alkoxy or NHR¹⁰;
R¹⁰ is hydrogen, C₁₋₅ alkyl or C₁₋₅ alkoxy;
and pharmaceutically acceptable salts or esters thereof.

40. The method of claim 38 wherein the compound of formula (C) is

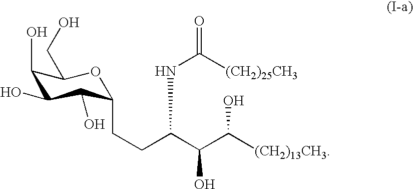

41. The method of claim 38 wherein the compound of formula (C) is

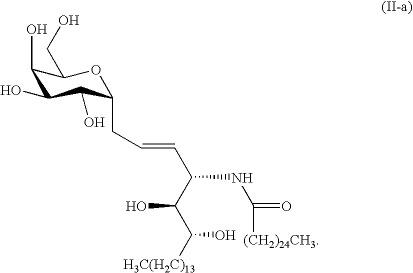

42. The method of claim 38 wherein the compound of formula (C) is

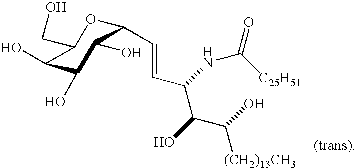

43. The method of claim 38 wherein the compound of formula (C) is

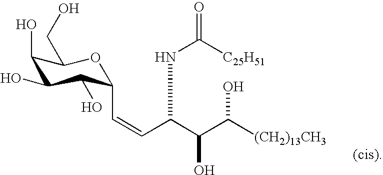

44. The method of claim 38 wherein the compound of formula (C) is

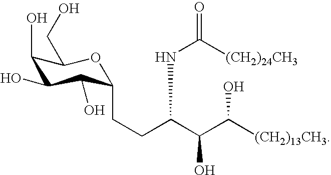

45. A method of synthesizing a glycolipid comprising the steps of:
(a) reacting a compound of Formula: 11: 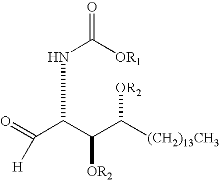
to form a compound of Formula 1: 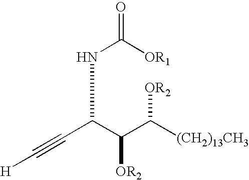
wherein R¹ is CBZ or t.-Boc; and
R² is joined to form a 5-7 member heterocyclic ring, optionally substituted with alkyl; and
(b) further reacting the compound of Formula 1 to form a compound of Formula Y: 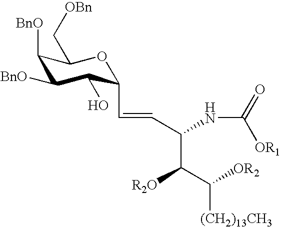
wherein R¹ is CBZ or t.-Boc; and
R² is joined to form a 5-7 member heterocyclic ring, optionally substituted with alkyl;
and pharmaceutically acceptable salts or esters thereof.

46. A method of synthesizing a glycolipid comprising the steps of:
(a) reacting a compound of Formula 1: 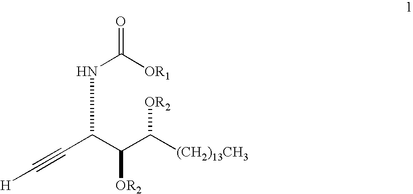
to form a compound of Formula 2: 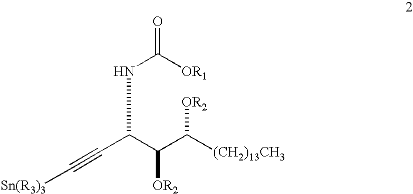
wherein R¹ is CBZ or t.-Boc; and
R² is joined to form a 5-7 member heterocyclic ring, optionally substituted with alkyl; and
(b) further reacting the compound of Formula 2 to form a compound of Formula 3: 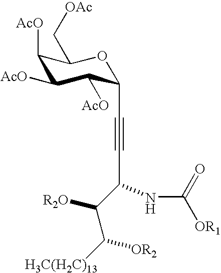
wherein R¹ is CBZ or t.-Boc; and
R² is joined to form a 5-7 member heterocyclic ring, optionally substituted with alkyl; and
(c) further reacting the compound of Formula 3 to form a compound of Formula 4: 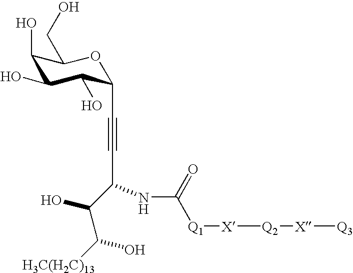
wherein Q¹ is optionally present and is a C₁₋₁₀ straight or branched chain alkylene, alkenylene, or alkynylene;
X′ is optionally present and is O, S or NR⁸;
Q² is optionally present and is a C₁₋₁₀ straight or branched chain alkylene, alkenylene or alkynylene;
X″ is optionally present and is O, S or NR⁸;
Q³ is a straight or branched chain C₁₋₁₀ alkyl, alkenyl or alkynyl, or is hydrogen,
wherein each Q¹, Q² or Q³ is optionally substituted with hydroxyl, halogen, cyano, nitro, SO₂, NHR⁸, or C(═O)—R⁹; and wherein
R⁸ is hydrogen, C₁₋₅ alkyl, C₁₋₅ alkoxy, halogen, cyano, nitro, SO₂ or C(═O)—R⁹;
R⁹ is hydrogen, C₁₋₅ alkyl, C₁₋₅ alkoxy or NHR¹⁰;
R¹⁰ is hydrogen, C₁₋₅ alkyl or C₁₋₅ alkoxy;
and pharmaceutically acceptable salts or esters thereof.
